SEQUENCE LISTING (10 sequences)

SEQ ID NO: 1 - protein, 21 aa, Homo sapiens
GIVEQCCTSICSLYQLENYCN

SEQ ID NO: 2 - protein, 30 aa, Homo sapiens
FVNQHLCGSHLVEALYLVCGERGFFYTPKT

SEQ ID NO: 3 - protein, 21 aa, Artificial Sequence
  Insulin A Chain
  MISC_FEATURE (2)..(2): Xaa is isoleucine or threonine
  MISC_FEATURE (3)..(3): Xaa is valine, glycine, or leucine
  MISC_FEATURE (8)..(8): Xaa is threonine or histidine
  MISC_FEATURE (17)..(17): Xaa is glutamic acid or glutamine
  MISC_FEATURE (19)..(19): Xaa is tyrosine, 4-methoxy- phenylalanine, alanine, or 4-amino phenylalanine
  MISC_FEATURE (21)..(21): Xaa is asparagine or glycine
GXXEQCCXSICSLYQLXNXCX

SEQ ID NO: 4 - protein, 25 aa, Artificial Sequence
  Insuline B chain
  MISC_FEATURE (1)..(1): X25 is histidine or threonine
  MISC_FEATURE (5)..(5): X29 is alanine, glycine or serine
  MISC_FEATURE (6)..(6): X30 is histidine, aspartic acid, glutamic acid, homocysteic acid, or cysteic acid
  MISC_FEATURE (21)..(21): X27 is phenylalanine or aspartic acid
  MISC_FEATURE (24)..(24): X31 is aspartic acid, proline or lysine
  MISC_FEATURE (25)..(25): X32 is proline or lysine, with the proviso that at least one of X31 or X32 is lysine
XLCGXXLVEALYLVCGERGFXYTXX

SEQ ID NO: 5 - protein, 32 aa, Artificial Sequence
  Insuline B chain
  MISC_FEATURE (1)..(1): X22 is phenylalanine or desamino-phenylalanine
  MISC_FEATURE (5)..(5): X25 is histidine or threonine
  MISC_FEATURE (6)..(6): X26 is glycine or leucine
  MISC_FEATURE (9)..(9): X29 is alanine, glycine, or serine
  MISC_FEATURE (10)..(10): X30 is histidine, aspartic acid, glutamic acid, homocysteic acid, or cysteic acid
  MISC_FEATURE (25)..(25): X27 is phenylalanine or aspartic acid
  MISC_FEATURE (28)..(28): X31 is aspartic acid, proline, or lysine
  MISC_FEATURE (29)..(29): X32 is lysine or proline
  MISC_FEATURE (30)..(30): X33 is threonine, alanine, or absent
  MISC_FEATURE (31)..(31): X34 is arginine or absent
  MISC_FEATURE (32)..(32): X35 is is arginine or absent;  With the proviso at least one of X31 or X32 is lysine
XVNQXXCGXXLVEALYLVCGERGFXYTXXXXX

SEQ ID NO: 6 - protein, 30 aa, Artificial Sequence
  Insulin lispro B chain
FVNQHLCGSHLVEALYLVCGERGFFYTKPT

SEQ ID NO: 7 - protein, 21 aa, Artificial Sequence
  Insulin glargine A chain
GIVEQCCTSICSLYQLENYCG

SEQ ID NO: 8 - protein, 32 aa, Artificial Sequence
  Insulin glargine B chain
FVNQHLCGSHLVEALYLVCGERGFFYTPKTRR

SEQ ID NO: 9 - protein, 30 aa, Artificial Sequence
  Insulin aspart B chain
FVNQHLCGSHLVEALYLVCGERGFFYTDKT

SEQ ID NO: 10 - protein, 29 aa, Artificial Sequence
  Bdes30
FVNQHLCGSHLVEALYLVCGERGFFYTPK

(19) United States
(12) Patent Application Publication
(10) Pub. No.: US 2024/0294596 A1
(43) Pub. Date: Sep. 5, 2024

(54) INSULIN RECEPTOR PARTIAL AGONISTS

(71) Applicant: Merck Sharp & Dohme LLC, Rahway, NJ (US)

(72) Inventors: Dmitri A. Pissarnitski, Scotch Plains, NJ (US); Danqing Feng, Green Brook, NJ (US); Pei Huo, Millburn, NJ (US); Ahmet Kekec, Hoboken, NJ (US); Songnian Lin, Holmdel, NJ (US); Ravi Nargund, Skillman, NJ (US); Zhicai Shi, Monmouth Junction, NJ (US); Zhicai Wu, Montvale, NJ (US); Lin Yan, East Brunswick, NJ (US); Yuping Zhu, Basking Ridge, NJ (US)

(73) Assignee: Merck Sharp & Dohme LLC, Rahway, NJ (US)

(21) Appl. No.: 18/041,815

(22) PCT Filed: Sep. 7, 2021
(86) PCT No.: PCT/US21/49277

§ 371 Date: Feb. 16, 2023

Related U.S. Application Data
(60) Provisional application No. 63/076,142, filed on Sep. 9, 2020.

(51) Int. Cl. C07K 14/62; A61K 38/28; A61K 45/06; A61P 3/10; C07K 1/107

(52) U.S. Cl. CPC C07K 14/62; A61K 38/28; A61K 45/06; A61P 3/10; C07K 1/1077

(57) ABSTRACT

Insulin dimers and insulin analog dimers that act as partial agonists at the insulin receptor are disclosed.

### BACKGROUND OF THE INVENTION

The present invention relates to insulin dimers and insulin analog dimers that act as partial agonists at the insulin receptor.

### REFERENCE TO SEQUENCE LISTING SUBMITTED ELECTRONICALLY

The sequence listing of the present application is submitted electronically via EFS-Web as an ASCII formatted sequence listing with a file name 25094-US-PCT-SEQLIST.txt, creation date of Oct. 12, 2023, and a size of 6,163 bytes. This sequence listing is part of the specification and is herein incorporated by reference in its entirety.

### DESCRIPTION OF RELATED ART

Insulin is the most effective anti-diabetic therapy for glycemic control in diabetic patients, but high hypoglycemia risk limits its treatment efficacy. The key reason that diabetic patients on insulin are not attaining their HbA1C goal is because administration doses are often intentionally lowered to avoid potentially life-threatening hypoglycemia. To improve the narrow therapeutic index (TI) of insulin may allow for further lowering glucose level to attain glycemic control with lower hypoglycemic risk, reducing health care cost associated with hypoglycemia treatment. Among various active research areas of developing insulins with improved TI, covalently linked insulin dimers have been reported in the literature to function as partial agonists of insulin receptor. It is believed that partial agonism of the insulin receptor by these covalent dimers may elicit a desired submaximal activation of the insulin receptor while it may also reduce overactivation of the insulin receptor by excess amount of the endogenous insulin, leading to an increased therapeutic index in vivo.

Insulin is an essential therapy for type 1 diabetes mellitus (T1DM) patients and many type 2 mellitus diabetics (T2DMs), prescribed to close to one third of U.S. patients among all anti-diabetic drug users in the past decade. The worldwide market for insulins was US$20.4 billion in 2013 and is growing at a faster rate than all other anti-diabetic agents combined. However, challenges of current insulin therapies, including narrow TI to hypoglycemia and body weight gain, limit their wider adoption and potential for patients to achieve ideal glycemic control.

In addition to prandial insulin secretion in response to meals, the pancreas releases insulin at a “basal” rate, governed largely by plasma glucose levels to maintain appropriate fasting glucose regulation. This is achieved mainly by controlling hepatic glucose release, through endogenous insulin's hepato-preferring action. Modern insulin analogs include rapid acting and basal insulins, as well as mixtures of these two.

Rapid-acting insulin analogs (RAA) are developed to control post-prandial hyperglycemia while insulins with extended duration of action regulate basal glucose levels. Long-acting insulins are used by all T1DM (in 25 combination with prandial injections) and the majority of T2DM patients start their insulin therapy from a basal product. Basal insulin consumption is growing rapidly as the worldwide diabetes population (particularly T2DM) soars.

Despite continuous development efforts over the past several decades. available long-acting insulins are still not optimized compared to physiological basal insulin. This is partially because major focus was on improving PK flatness of these analogs but not fixing the relative over-insulinization of peripheral tissues, which contributes to increased hypoglycemia risk. As a result, hypoglycemia remains a key medical risk with huge burden on patients and causes significant morbidity and mortality.

### BRIEF SUMMARY OF THE INVENTION

The present invention provides compounds comprising two insulin molecules covalently linked to form an insulin molecule dimer that may activate the insulin receptor with regular insulin-like potency but with reduced maximum activity. These compounds are insulin receptor partial agonists (IPRAs): they behave like other insulin analogs to lower glucose effectively but with lower risk of hypoglycemia.

Provided are insulin receptor partial agonist covalent insulin dimers formulated as novel and transformative basal insulins (once daily administration) that manifest an improved therapeutic index (TI) over current standard of care (SOC) basal insulins. In one embodiment, the IPRAs of the present invention may lower glucose effectively with reduced risk of hypoglycemia in diabetic minipig and has the property of a once daily (QD) basal insulin. The improved TI may empower practitioners to more aggressively dose IRPAs of the present invention to achieve target goals for control of fasting glucose. Tight control of fasting glucose and HbA1c by an IRPA may allow it to serve as 1) a stand-alone long-acting insulin with an enhanced efficacy and safety profile in T2DM and 2) an improved foundational basal insulin in T1DM (and some T2DM) for use with additional prandial rapid-acting insulin analogs (RAA) doses. Thus, the present invention provides the following embodiments.

The present invention provides an insulin dimer comprising an epsilon (ε)-amine of a lysine group on the B chain of a first insulin having a first A-chain polypeptide and first B-chain polypeptide and an α-amino group of A1′ residue of a second insulin molecule having a second A′-chain polypeptide and second B′-chain polypeptide conjugated together by a bifunctional linker moiety selected from Linker 1, Linker 2, Linker 3, Linker 4, Linker 5, Linker 6, Linker 7, Linker 8, Linker 9, Linker 10, Linker 11, Liner 12, Linker 13, Linker 14, Linker 15, Linker 16, Linker 17, Linker 18, Linker 19, Linker 20, Linker 21, Linker 22, and Linker 23.

An aspect of this invention is realized wherein an insulin dimer comprising an epsilon (ε)-amine of the B-29 or B28 lysine on the B chain of a first insulin molecule having a first A-chain polypeptide and second B-chain polypeptide and an α-amino group of A1 Gly residue of a second insulin molecule having a second A′-chain polypeptide and second B′-chain polypeptide conjugated together by a bifunctional linker moiety selected from Linker 1, Linker 2, Linker 3, Linker 4, Linker 5, Linker 6, Linker 7, Linker 8, Linker 9, Linker 10, Linker 11, Liner 12, Linker 13, Linker 14, Linker 15, Linker 16, Linker 17, Linker 18, Linker 19, Linker 20, Linker 21, Linker 22, and Linker 23, wherein the A-chain, A′-chain, B-chain and B′-chain are illustrated, for example, in Formula I.

In a further aspect of the insulin dimer, the first and second insulin or insulin analog heterodimers are the same or wherein the first and second insulin or insulin analog heterodimers are different.

In particular aspects of the insulin dimer, the linker moiety covalently links the first insulin or insulin analog heterodimer and the second insulin or insulin analog heterodimer via the epsilon amino group of a lysine residue at or near the carboxy terminus of the first insulin or insulin analog heterodimer's B chain polypeptides and the alpha amino group of a glycine at the amino terminus of the second insulin or insulin analog heterodimer's A chain.

In particular aspects of the insulin dimer, at least one of the first or second A-chain or B-chain polypeptides is conjugated at its N-terminal amino acid to a capping group or at least the N-terminal amino acids of the first insulin heterodimer molecule are conjugated to a capping group or the N-terminal amino acids of both the first insulin heterodimer and second insulin heterodimer are conjugated to a capping group.

Another aspect of the insulin dimer is realized when capping groups include acyl moieties comprising carbamates, PEG-containing chains, sugar-containing groups, carboxylic acid containing groups, phosphonate groups, aryl groups (including but not limited to unsubstituted and substituted phenyl groups), and aromatic or non-aromatic heterocycles (including but not limited to morpholinyl, tetrahydrofuranyl, and fused versions of thereof), or a mixture thereof. A subembodiment of this aspect of the invention is realized when the capping group is an acyl moiety bearing one or more carbamates. Another subembodiment of this aspect of the invention is realized when the capping group is an acyl moiety bearing one or more PEG-containing chains. A subembodiment of this aspect of the invention is realized when the PEG in the PEG-containing chains is selected from PEG2 through PEG25. A subembodiment of this aspect of the invention is realized when the capping group is an acyl moiety bearing one or more sugar-containing groups. A subembodiment of this aspect of the invention is realized when the capping group is an acyl moiety bearing one or more carboxylic acid containing groups. A subembodiment of this aspect of the invention is realized when the capping group is an acyl moiety bearing one or more amines. A subembodiment of this aspect of the invention is realized when the capping group is an acyl moiety bearing one or more amides. A subembodiment of this aspect of the invention is realized when the capping group is an acyl moiety bearing one or more hydroxyls. A subembodiment of this aspect of the invention is realized when the capping group is an acyl moiety bearing one or more phosphonates. A subembodiment of this aspect of the invention is realized when the capping group is an acyl moiety bearing one or more heterocycle groups.

Another aspect of the insulin dimer is realized when the capping group is a linear or branch C₁₋₆ alkyl, or has the general formula RC(O)—, where R is:
a) a peptide,
b) PEG,
c) linear or branched C₁₋₆ alkyl chain,
d) R′NH—, or
e) R′O—,
   wherein R′ is H (when R is R′NH—), peptide, PEG, or linear or branched alkyl chain, and wherein each said peptide, PEG and linear or branched alkyl may be unsubstituted or substituted with 1 to 3 groups selected from amino-, phosphono-, hydroxy-, carboxylic acid, amino acid, PEG, and saccharides.

In aspects of this invention the capping group is, for example dimethyl, isobutyl, or is a group RC(O) that may be exemplified as acetyl, phenylacetyl, isobutyl, methoxyacetyl, 2-(carboxymethoxy)acetyl, 2-[bis(carboxymethylamino)]acetyl, glutaryl, trifluoroacetyl, glycyl, aminoethylglucose AEG-C6, PEG (e.g., PEG1, PEG2, PEG3, PEG4, PEG5, PEG8, PEG24,), phosphonoacetyl, morpholinohexanoyl, and alkoxycarbonyl.

A particular aspect of the insulin dimer is realized when the capping group is selected from Capping Group 1, 2, 3, 4, 5, 6, and 7, or a mixture thereof from Table II. A subembodiment of this aspect of the invention is realized when at least one of the capping group of Table II is 1. A further subembodiment of this aspect of the invention is realized when at least one of the capping group of Table II is 2. A further subembodiment of this aspect of the invention is realized when at least one of the capping group of Table II is 3. A further subembodiment of this aspect of the invention is realized when at least one of the capping group of Table II is 4. A further subembodiment of this aspect of the invention is realized when at least one of the capping group of Table II is 5. A further subembodiment of this aspect of the invention is realized when at least one of the capping group of Table II is 6. A further subembodiment of this aspect of the invention is realized when at least one of the capping group of Table II is 7.

The term AEG-C6 is depicted as

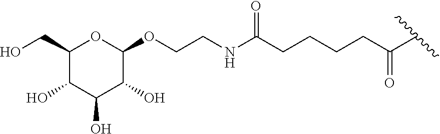 [SMILES: *C(=O)CCCCC(=O)NCCO[C@@H]1O[C@H](CO)[C@@H](O)[C@H](O)[C@H]1O]

In particular aspects of the insulin dimer, the first insulin and the second insulin heterodimers are independently native human insulin, insulin lispro, insulin aspart, desB30 insulin, or insulin glargine.

The present invention further provides a composition comprising a first insulin or insulin analog heterodimer and a second insulin or insulin analog heterodimer each heterodimer including an A-chain polypeptide and a B-chain polypeptide, wherein the A-chain polypeptide and the B-chain polypeptide are linked together through interchain disulfide bonds; wherein the first and second insulin or insulin analog heterodimers are covalently linked together through a linking moiety joining the B-chain of the first insulin analog at or near carboxy terminus with the A′-chain of the second insulin analog at or close to its amino terminus; wherein the linking moiety is selected from the group consisting of Linking moiety 1, 2, 3, 4, 5, 6, 7, 8, 9, 10, 11, 12, 13, 14, 15, 16, 17, 18, 19, 20, 21, 22, and 23; wherein the insulin is recombinant human insulin and the insulin analog is selected from the group consisting of insulin lispro, insulin aspart, desB30 insulin and insulin glargine; and wherein the amino terminus of at least one of the A-chain polypeptides and the B-chain polypeptides of the first insulin polypeptide or second insulin polypeptide is covalently linked to a capping group.

In particular aspects of the insulin dimer, the first and second insulin or insulin analog heterodimers are the same or wherein the first and second insulin or insulin analog heterodimers are different.

In particular aspects of the insulin dimer, the linking moiety covalently links the first insulin or insulin analog heterodimer and the second insulin or insulin analog heterodimer via the epsilon amino group of a lysine residue at or near the carboxy terminus of the first insulin or insulin analog heterodimer's B chain polypeptides and the alpha amino group of a glycine at the amino terminus of the second insulin or insulin analog heterodimer's A chain.

Exemplary insulin dimers of the present invention are represented by Formula I:

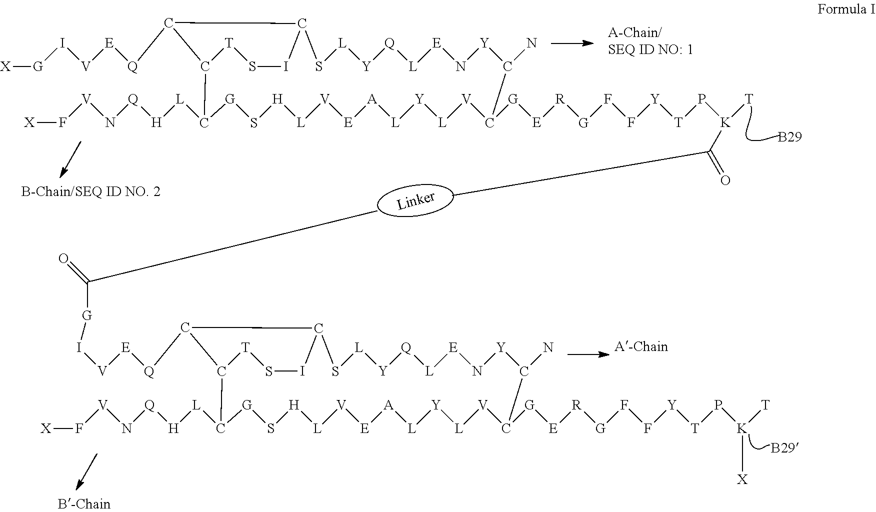

wherein A1, B1. and B1′ and B29′ sites are blocked by small capping groups represented by X, a first insulin heterodimer molecule having a first A-chain polypeptide and first B-chain polypeptide and a second insulin heterodimer having a second A′-chain polypeptide and second B′-chain polypeptide that is conjugated together at the B29 of the first and A1′ of the second heterodimer, respectively, by a bifunctional linking group represented by Linker, the A-chain and A′-chain peptides have the amino acid sequence shown in SEQ ID NO: 1 and the B-chain and B′-chain peptides have the amino acid sequence shown in SEQ ID NO: 2, and wherein the cysteine residues at positions 6 and 11 of the A chain are linked in a disulfide bond, the cysteine residues at position 7 of the A chain and position 7 of the B chain are linked in a disulfide bond, and the cysteine residues at position 20 of the A chain and 19 of the B chain are linked in a disulfide bond.

The present invention further provides a composition comprising an insulin dimer selected from the group of consisting of Dimers 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, and 33.

The present invention further provides a method for treating diabetes comprising administering to an individual with diabetes a therapeutically effective amount of a composition comprising the insulin receptor partial agonist of any one of insulin dimers. In particular aspects the diabetes is Type 1 diabetes, Type 2 diabetes, or gestational diabetes.

The present invention further provides a composition for the treatment of diabetes comprising the any one of the above insulin dimers. In particular aspects the diabetes is Type 1 diabetes, Type 2 diabetes, or gestational diabetes.

The present invention further provides for the use of any one of the above the insulin dimers for the manufacture of a medicament for the treatment of diabetes. In particular aspects the diabetes is Type 1 diabetes, Type 2 diabetes, or gestational diabetes.

### Definitions

Insulin—as used herein, the term means the active principle of the pancreas that affects the metabolism of carbohydrates in the animal body and which is of value in the treatment of diabetes mellitus. The term includes synthetic and biotechnologically derived products that are the same as, or similar to, naturally occurring insulins in structure, use, and intended effect and are of value in the treatment of diabetes mellitus. The term is a generic term that designates the 51 amino acid heterodimer comprising the A-chain peptide having the amino acid sequence shown in SEQ ID NO: 1 and the B-chain peptide having the amino acid sequence shown in SEQ ID NO: 2, wherein the cysteine residues a positions 6 and 11 of the A chain are linked in a disulfide bond, the cysteine residues at position 7 of the A chain and position 7 of the B chain are linked in a disulfide bond, and the cysteine residues at position 20 of the A chain and 19 of the B chain are linked in a disulfide bond.

Insulin analog or analogue—the term as used herein includes any heterodimer analogue or single-chain analogue that comprises one or more modification(s) of the native A-chain peptide and/or B-chain peptide. Modifications include but are not limited to substituting an amino acid for the native amino acid at a position selected from A4, A5, A8, A9, A10, A12, A13, A14, A15, A16, A17, A18, A19, A21, B1, B2, B3, B4, B5, B9, B10, B13, B14, B15, B16, B17, B18, B20, B21, B22, B23, B26, B27, B28, B29, and B30; deleting any or all of positions B1-4 and B26-30; or conjugating directly or by a polymeric or non-polymeric linker one or more acyl, polyethylglycine (PEG), or saccharide moiety (moieties); or any combination thereof. As exemplified by the N-linked glycosylated insulin analogues disclosed herein, the term further includes any insulin heterodimer and single-chain analogue that has been modified to have at least one N-linked glycosylation site and in particular, embodiments in which the N-linked glycosylation site is linked to or occupied by an N-glycan. Examples of insulin analogues include but are not limited to the heterodimer and single-chain analogues disclosed in published international application WO20100080606, WO2009/099763, and WO2010080609, the disclosures of which are incorporated herein by reference. Examples of single-chain insulin analogues also include but are not limited to those disclosed in published International Applications WO9634882, WO95516708, WO2005054291, WO2006097521, WO2007104734, WO2007104736, WO2007104737, WO2007104738, WO2007096332, WO2009132129; U.S. Pat. Nos. 5,304,473, 6,630,348 and 8,273,361; and Kristensen et al., Biochem. J. 305: 981-986 (1995), the disclosures of which are each incorporated herein by reference.

The term further includes single-chain and heterodimer polypeptide molecules that have little or no detectable activity at the insulin receptor but which have been modified to include one or more amino acid modifications or substitutions to have an activity at the insulin receptor that has at least 1%, 10%, 50%, 75%, or 90% of the activity at the insulin receptor as compared to native insulin and which further includes at least one N-linked glycosylation site. In particular aspects, the insulin analogue is a partial agonist that has less than 80% (or 70%) activity at the insulin receptor as does native insulin. These insulin analogues, which have reduced activity at the insulin growth hormone receptor and enhanced activity at the insulin receptor, include both heterodimers and single-chain analogues.

Single-chain insulin or single-chain insulin analog as used herein, the term encompasses a group of structurally-related proteins wherein the A-chain peptide or functional analogue and the B-chain peptide or functional analogue are covalently linked by a peptide or polypeptide of 2 to 35 amino acids or non-peptide polymeric or non-polymeric linker and which has at least 1%, 10%, 50%, 75%, or 90% of the activity of insulin at the insulin receptor as compared to native insulin. The single-chain insulin or insulin analogue further includes three disulfide bonds: the first disulfide bond is between the cysteine residues at positions 6 and 11 of the A-chain or functional analogue thereof, the second disulfide bond is between the cysteine residues at position 7 of the A-chain or functional analogue thereof and position 7 of the B-chain or functional analogue thereof, and the third disulfide bond is between the cysteine residues at position 20 of the A-chain or functional analogue thereof and position 19 of the B-chain or functional analogue thereof.

Insulin dimer—as used herein, the term refers to a dimer comprising two insulin molecules (also referred to as heterodimers) linked together via their respective lysine residues at or near the C-terminus of their respective B-chain polypeptides (i.e., the B29 Lysine) via a linking moiety as disclosed herein.

Boc—as used herein, the term refers to tert-butoxycarbonyl.

Heterocycles—as used herein, the term refers to a nonaromatic 3-10 membered monocyclic, 8-12 membered bicyclic, or 11-14 membered tricyclic ring system having 1-3 heteroatoms of monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively). The heterocyclyl groups herein described may also contain fused rings. Fused rings are rings that share a common carbon-carbon bond or a common carbon atom (e.g., spiro-fused rings). Examples of heterocyclyl include, but are not limited to tetrahydrofuranyl, tetrahydropyranyl, piperidinyl, morpholino, pyrrolinyl and pyrrolidinyl.

Amino acid modification—as used herein, the term refers to a substitution of an amino acid, or the derivation of an amino acid by the addition and/or removal of chemical groups to/from the amino acid, and includes substitution with any of the 20 amino acids commonly found in human proteins, as well as atypical or non-naturally occurring amino acids. Commercial sources of atypical amino acids include Sigma-Aldrich (Milwaukee, WI), ChemPep Inc. (Miami, FL), and Genzyme Pharmaceuticals (Cambridge, MA). Atypical amino acids may be purchased from commercial suppliers, synthesized de novo, or chemically modified or derivatized from naturally occurring amino acids.

Amino acid substitution—as used herein refers to the replacement of one amino acid residue by a different amino acid residue.

Conservative amino acid substitution—as used herein, the term is defined herein as exchanges within one of the following five groups:
I. Small aliphatic, nonpolar or slightly polar residues:
   Ala, Ser, Thr, Pro, Gly;
II. Polar, negatively charged residues and their amides:
   Asp, Asn, Glu, Gln, cysteic acid and homocysteic acid;
III. Polar, positively charged residues:
   His, Arg, Lys; Ornithine (Orn)
IV. Large, aliphatic, nonpolar residues:
   Met, Leu, Ile, Val, Cys, Norleucine (Nle), homocysteine
V. Large, aromatic residues:
   Phe, Tyr, Trp, acetyl phenylalanine

Treat—As used herein, the term “treat” (or “treating”, “treated”, “treatment”, etc.) refers to the administration of an IRPA of the present disclosure to a subject in need thereof with the purpose to alleviate, relieve, alter, ameliorate, improve or affect a condition (e.g., diabetes), a symptom or symptoms of a condition (e.g., hyperglycemia), or the predisposition toward a condition. For example, as used herein the term “treating diabetes” will refer in general to maintaining glucose blood levels near normal levels and may include increasing or decreasing blood glucose levels depending on a given situation.

The term AEG-C6 is depicted as:

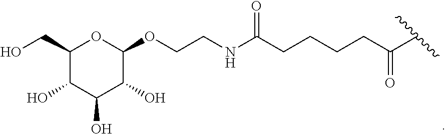 [SMILES: *C(=O)CCCCC(=O)NCCO[C@@H]1O[C@H](CO)[C@@H](O)[C@H](O)[C@H]1O]

Pharmaceutically acceptable carrier—as used herein, the term includes any of the standard pharmaceutical carriers, such as a phosphate buffered saline solution, water, emulsions such as an oil/water or water/oil emulsion, and various types of wetting agents suitable for administration to or by an individual in need. The term also encompasses any of the agents approved by a regulatory agency of the US Federal government or listed in the US Pharmacopeia for use in animals, including humans.

Pharmaceutically acceptable salt—as used herein, the term refers to salts of compounds that retain the biological activity of the parent compound, and which are not biologically or otherwise undesirable. Many of the compounds disclosed herein are capable of forming acid and/or base salts by virtue of the presence of amino and/or carboxyl groups or groups similar thereto.

Pharmaceutically acceptable base addition salts can be prepared from inorganic and organic bases. Salts derived from inorganic bases, include by way of example only, sodium, potassium, lithium, ammonium, calcium, zinc, and magnesium salts. Salts derived from organic bases include, but are not limited to, salts of primary, secondary and tertiary amines.

Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like.

Effective or therapeutically effective amount—as used herein refers to a nontoxic but sufficient amount of an insulin analog to provide the desired effect. For example one desired effect would be the prevention or treatment of hyperglycemia. The amount that is “effective” will vary from subject to subject, depending on the age and general condition of the individual, mode of administration, and the like. Thus, it is not always possible to specify an exact “effective amount.” It is not always possible to determine the optimal effective amount prior to administration to or by an individual in need thereof. However, an appropriate “effective” amount in any individual case may be determined by one of ordinary skill in the art using routine experimentation.

Parenteral—as used herein, the term means not through the alimentary canal but by some other route such as intranasal, inhalation, subcutaneous, intramuscular, intraspinal, or intravenous.

### BRIEF DESCRIPTION OF THE DRAWINGS

FIG. 1 shows the change in plasma glucose in diabetic minipigs over time for Dimers 2 and 5 compared to recombinant human insulin (RHI). Dimers and RHI were administered at 0.69 nmol/kg.

FIG. 2 shows the change in plasma glucose in diabetic minipigs over time for Dimers 7, 11, and 20 compared to recombinant human insulin (RHI). Dimers and RHI were administered at 0.69 nmol/kg.

FIG. 3 shows the change in plasma glucose in diabetic minipigs over time for Dimers 22 and 31 compared to recombinant human insulin (RHI). Dimers and RHI were administered at 0.69 nmol/kg.

### DETAILED DESCRIPTION OF THE INVENTION

The present invention provides compounds comprising two insulin molecules covalently linked to form a covalently linked insulin dimer that may activate the insulin receptor with regular insulin-like potency and reduced maximum activity. These compounds are insulin receptor partial agonists (IRPA): they behave like other insulin analogs to lower glucose effectively but with lower risk of hypoglycemia.

Insulin dimers have been disclosed in Brandenburg et al. in U.S. Pat. No 3,907,763 (1973); Tatnell et al., Biochem J. 216: 687-694 (1983); Shüttler and Brandenburg, Hoppe-Seyler's Z. Physiol. Chem. 363, 317-330, 1982; Weiland et al., Proc Natl. Acad. Sci. (USA) 87: 1154-1158 (1990); Deppe et al., Naunyn-Schmiedeberg's Arch Pharmacol (1994) 350:213-217; Brandenburg and Havenith in U.S. Pat. No. 6,908,897(B2) (2005); Knudsen et al., PLOS ONE 7: e51972 (2012); DiMarchi et al in WO2011/159895; DiMarchi et al. in WO2014/052451; and Herrera et al., WO2014141165. More recently, insulin dimers have been described in Brant-Synthesis and Characterization of Insulin Receptor Partial Agonists as a Route to Improved Diabetes Therapy, Ph.D. Dissertation. Indiana University (April 2015) and Zaykov and DiMarchi. Poster P2/2-Exploration of the structural and mechanistic basis for partial agonism of insulin dimers, American Peptide Symposium, Orlando FL (Jun. 20-25 (2015). However, the inventors of the instant invention have discovered that the level of insulin activity and partial agonist activity of the dimers is a function of the dimeric structure, the sequence of the insulin analog, the length of the dimerization linker, and the site of dimerization that connects the two insulin polypeptides. The inventors have discovered that the insulin dimers of the present invention have reduced risk of promoting hypoglycemia when administered in high doses than native insulin or other insulin analogs when administered at high doses.

The present invention provides partial agonist covalently-linked insulin dimers formulated as a novel and transformative basal insulin (once daily administration) that manifests improved therapeutic index (TI) over current standard of care (SOC) basal insulins. These molecules may lower glucose effectively with reduced risk of hypoglycemia in diabetic minipig and have the property of a once daily (QD) basal insulin. The improved TI may enable practitioners to more aggressively dose IRPA insulin dimer to achieve target goals for control of fasting glucose. Tight control of fasting glucose and HbA1c may allow these molecules to serve as 1) a stand-alone long-acting insulin with an enhanced efficacy and safety profile in Type 2 diabetes mellitus (T2DM) and 2) an improved foundational basal insulin in Type 1 diabetes mellitus (T1DM) (and some T2DM) for use with additional prandial rapid-acting insulin analogs (RAA) doses.

An ideal long-acting insulin provides continuous control of fasting glucose in diabetics with highly stable and reproducible PK/PD. However, currently available basal insulins, even those with improved stability and reproducibility of PK/PD continue to have a narrow therapeutic index and hypoglycemia incidents increase as glucose levels approach euglycemia target. This can often lead to underdosing to avoid hypoglycemia. Treatment with an IRPA of the present invention is expected to be safer with respect to hypoglycemia due to reduced maximal effect of the drug.

### Insulin A and B Chains

Disclosed herein are insulin or insulin analog dimers that have insulin receptor agonist activity. The level of insulin activity of the dimers is a function of the dimeric structure, the sequence of the insulin analog, the length of the dimerization linker, and the site of dimerization that connects the two insulin polypeptides. The insulin polypeptides of the present invention may comprise the native A and B chain sequences of human insulin (SEQ ID NOs: 1 and 2, respectively) or any of the known analogs or derivatives thereof that exhibit insulin agonist activity when linked to one another in a heteroduplex. Such analogs include, for example, proteins that having an A-chain and a B-chain that differ from the A-chain and B-chain of human insulin by having one or more amino acid deletions, one or more amino acid substitutions, and/or one or more amino acid insertions that do not destroy the insulin activity of the insulin analog.

One type of insulin analog, “monomeric insulin analog,” is well known in the art. These are fast-acting analogs of human insulin, including, for example, insulin analogs wherein:
(a) the amino acyl residue at position B28 is substituted with Asp, Lys, Leu, Val, or Ala, and the amino acyl residue at position B29 is Lys or Pro;
(b) the amino acyl residues at any of positions B27 and B30 are deleted or substituted with a nonnative amino acid.

In one embodiment an insulin analog is provided comprising an Asp substituted at position B28 (e.g., insulin aspart (NOVOLOG); see SEQ ID NO:9) or a Lys substituted at position 28 and a proline substituted at position B29 (e.g., insulin lispro (HUMALOG); see SEQ ID NO:6). Additional monomeric insulin analogs are disclosed in Chance, et al., U.S. Pat. No. 5,514,646; Chance, et al., U.S. patent application Ser. No. 08/255,297; Brems, et al., Protein Engineering, 5:527-533 (1992); Brange, et al., EPO Publication No. 214,826 (published Mar. 18, 1987); and Brange, et al., Current Opinion in Structural Biology, 1:934-940 (1991). These disclosures are expressly incorporated herein by reference for describing monomeric insulin analogs.

Insulin analogs may also have replacements of the amidated amino acids with acidic forms. For example, Asn may be replaced with Asp or Glu. Likewise, Gln may be replaced with Asp or Glu. In particular, Asn(A18), Asn(A21), or Asp(B3), or any combination of those residues, may be replaced by Asp or Glu. Also, Gln(A15) or Gln(B4), or both, may be replaced by either Asp or Glu.

As disclosed herein insulin single chain analogs are provided comprising a B chain and A chain of human insulin, or analogs or derivative thereof, wherein the carboxy terminus of the B chain is linked to the amino terminus of the A chain via a linking moiety. In one embodiment the A chain is amino acid sequence GIVEQCCTSICSL YQLENYCN (SEQ ID NO: 1 and the B chain comprises amino acid sequence FVNQHLCGSH LVEALYLVCGERGFFYTPKT (SEQ ID NO: 2) or a carboxy shortened sequence thereof having B30 deleted, and analogs of those sequences wherein each sequence is modified to comprise one to five amino acid substitutions at positions corresponding to native insulin positions selected from A5, A8, A9, A10, A14, A15, A17, A18, A21, B1, B2, B3, B4, B5, B9, B10, B13, B14, B20, B22, B23, B26, B27, B28, B29 and B30, with the proviso that at least one of B28 or B29 is lysine. In one embodiment the amino acid substitutions are conservative amino acid substitutions. Suitable amino acid substitutions at these positions that do not adversely impact insulin's desired activities are known to those skilled in the art, as demonstrated, for example, in Mayer, et al., Insulin Structure and Function, Biopolymers. 2007; 88(5):687-713, the disclosure of which is incorporated herein by reference.

In accordance with one embodiment the insulin analog peptides may comprise an insulin A chain and an insulin B chain or analogs thereof, wherein the A chain comprises an amino acid sequence that shares at least 70% sequence identity (e.g., 70%, 75%, 80%, 85%, 90%, 95%) over the length of the native peptide, with GIVEQCCTSICSLYQLENYCN (SEQ ID NO: 1) and the B chain comprises an amino acid sequence that shares at least 60% sequence identity (e.g., 60%, 65%, 70%, 75%, 80%, 85%, 90%, 95%) over the length of the native peptide, with FVNQHLCGSHL VEALYLVCGERGFFYTPKT (SEQ ID NO: 2) or a carboxy shortened sequence thereof having B30 deleted.

Additional amino acid sequences can be added to the amino terminus of the B chain or to the carboxy terminus of the A chain of the insulin polypeptides of the present invention. For example, a series of negatively charged amino acids can be added to the amino terminus of the B chain, including for example a peptide of 1 to 12, 1 to 10, 1 to 8 or 1 to 6 amino acids in length and comprising one or more negatively charged amino acids including for example glutamic acid and aspartic acid. In one embodiment the B chain amino terminal extension comprises 1 to 6 charged amino acids. In accordance with one embodiment the insulin polypeptides disclosed comprise a C-terminal amide or ester in place of a C-terminal carboxylate on the A chain.

In various embodiments, the insulin analog has an isoelectric point that has been shifted relative to human insulin. In some embodiments. the shift in isoelectric point is achieved by adding one or more arginine, lysine, or histidine residues to the N-terminus of the insulin A-chain peptide and/or the C-terminus of the insulin B-chain peptide. Examples of such insulin polypeptides include Arg^{A0}-human insulin, Arg^{B31}Arg^{B32}-human insulin, Gly^{A21}Arg^{B31}Arg^{B32}-human insulin, Arg^{A0}Arg^{B31}Arg^{B32}-human insulin, and Arg^{A0}Gly^{A21}Arg^{B31}Arg^{B32}-human insulin, Arg^{A0}Arg^{B31}Arg^{B32}-human insulin, and Arg^{A0}Gly^{A21}Arg^{B31}Arg^{B32}-human insulin. By way of further example, insulin glargine (LANTUS; see SEQ ID NOs: 7 and 8) is an exemplary long-acting insulin analog in which Asn^{A21} has been replaced by glycine, and two arginine residues have been covalently linked to the C-terminus of the B-peptide. The effect of these amino acid changes was to shift the isoelectric point of the molecule, thereby producing a molecule that is soluble at acidic pH (e.g., pH 4 to 6.5) but insoluble at physiological pH. When a solution of insulin glargine is injected into the muscle, the pH of the solution is neutralized and the insulin glargine forms microprecipitates that slowly release the insulin glargine over the 24 hour period following injection with no pronounced insulin peak and thus a reduced risk of inducing hypoglycemia.

This profile allows a once-daily dosing to provide a patient's basal insulin. Thus, in some embodiments, the insulin analog comprises an A-chain peptide wherein the amino acid at position A21 is glycine and a B-chain peptide wherein the amino acids at position B31 and B32 are arginine. The present disclosure encompasses all single and multiple combinations of these mutations and any other mutations that are described herein (e.g., Gly^{A21}-human insulin, Gly^{A21}Arg^{B31}-human insulin, Arg^{B31}Arg^{B32}-human insulin, Arg^{B31}-human insulin).

In particular aspects of the insulin receptor partial agonists, one or more amidated amino acids of the insulin analog are replaced with an acidic amino acid, or another amino acid. For example, asparagine may be replaced with aspartic acid or glutamic acid, or another residue. Likewise, glutamine may be replaced with aspartic acid or glutamic acid, or another residue. In particular, Asn^{A18}, Asn^{A21}, or Asn^{B3}, or any combination of those residues, may be replaced by aspartic acid or glutamic acid, or another residue. Gln^{A15} or Gln^{B4}, or both, may be replaced by aspartic acid or glutamic acid, or another residue. In particular aspects of the insulin receptor partial agonists, the insulin analogs have an aspartic acid, or another residue, at position A21 or aspartic acid, or another residue, at position B3, or both.

One skilled in the art will recognize that it is possible to replace yet other amino acids in the insulin analog with other amino acids while retaining biological activity of the molecule. For example, without limitation, the following modifications are also widely accepted in the art: replacement of the histidine residue of position B10 with aspartic acid (His^{B10} to Asp^{B10}); replacement of the phenylalanine residue at position B1 with aspartic acid (PheB1 to AspB1); replacement of the threonine residue at position B30 with alanine (ThrB30 to AlaB30); replacement of the tyrosine residue at position B26 with alanine (TyrB26 to AlaB26); and replacement of the serine residue at position B9 with aspartic acid (SerB9 to AspB9).

In various embodiments, the insulin analog has a protracted profile of action. Thus, in certain embodiments, the insulin analog may be acylated with a fatty acid. That is, an amide bond is formed between an amino group on the insulin analog and the carboxylic acid group of the fatty acid. The amino group may be the alpha-amino group of an N-terminal amino acid of the insulin analog, or may be the epsilon-amino group of a lysine residue of the insulin analog. The insulin analog may be acylated at one or more of the three amino groups that are present in wild-type human insulin or it may be acylated on lysine residue that has been introduced into the wild-type human insulin sequence. In particular aspects of the insulin receptor partial agonists, the insulin analog may be acylated at position B1, B1′, or both B1 and B1′.

Examples of insulin analogs can be found for example in published International Application WO9634882, WO95516708; WO20100080606, WO2009/099763, and WO2010080609, U.S. Pat. No. 6,630,348, and Kristensen et al., Biochem. J. 305: 981-986 (1995), the disclosures of which are incorporated herein by reference). In further embodiments, the in vitro glycosylated or in vivo N-glycosylated insulin analogs may be acylated and/or pegylated.

In particular aspects of the insulin dimer, each A-chain polypeptide independently comprises the amino acid sequence GX₂X₃EQCCX₈SICSLYQLX₁₇NX₁₉CX₂₃ (SEQ ID NO:3) and each B-chain polypeptide independently comprises the amino acid sequence X₂₅LCGX₂₉X₃₀LVEALYLVCGERGFX₂₇YTX₃₁X₃₂ (SEQ ID NO:4) or X₂₂VNQX₂₅X₂₆CGX₂₉X₃₀LVEALYLVCGERGFX₂₇YTX₃₁X₃₂X₃₃X₃₄X₃₅ (SEQ ID NO:5) wherein X₂ is isoleucine or threonine; X₃ is valine, glycine, or leucine; X₈ is threonine or histidine; X₁₇ is glutamic acid or glutamine; X₁₉ is tyrosine, 4-methoxy-phenylalanine, alanine, or 4-amino phenylalanine; X₂₃ is asparagine or glycine; X₂₂ is or phenylalanine and desamino-phenylalanine; X₂₅ is histidine or threonine; X₂₆ is leucine or glycine; X₂₇ is phenylalanine or aspartic acid; X₂₉ is alanine, glycine, or serine; X₃₀ is histidine, aspartic acid, glutamic acid, homocysteic acid, or cysteic acid; X₃₁ is aspartic acid, proline, or lysine; X₃₂ is lysine or proline; X₃₃ is threonine, alanine, or absent; X₃₄ is arginine or absent; and X₃₅ is arginine or absent; with the proviso at least one of X₃₁ or X₃₂ is lysine.

### Linking Moiety

The insulin dimers disclosed herein are formed between a first and second insulin polypeptide wherein each insulin polypeptide comprises an A chain and a B chain. The first and second insulin polypeptides may be two chain insulin analogs (i.e., wherein the A and B chains are linked only via inter-chain disulfide bonds between internal cysteine residues) wherein the first and second insulin polypeptides are linked to one another to form the dimer by a covalent bond or bifunctional linker. In accordance with one embodiment the first and second insulin polypeptides are linked to one another by a bifunctional linker joining the side chain of the B29 lysine of the B chain of the first insulin polypeptide to the alpha nitrogen of the A1′ glycine amino acid of the A′-chain of the second insulin polypeptide.

The following Table I shows exemplary linkers and linking moieties, which may be used to construct the dimers of the present invention. The bifunctional linker reagents shown comprise two 2,5-dioxopyrrolidin-1yl groups for conjugating to the epsilon amino group of the B29 lysine and alpha nitrogen of A1′ amino acid. Also shown are exemplary linking moieties of the invention.

TABLE I

| Linker | | |
|---|---|---|
| No. | Linker | Linking Moiety |
| 1 | 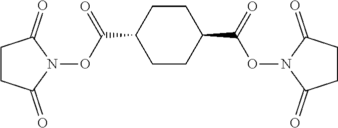 | 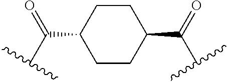 [SMILES: *C(=O)[C@H]1CC[C@H](C(*)=O)CC1] |
| 2 | 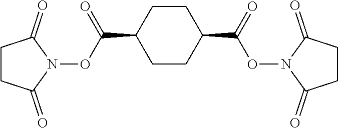 | 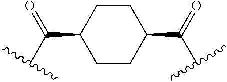 [SMILES: *C(=O)[C@H]1CC[C@@H](C(*)=O)CC1] |
| 3 | 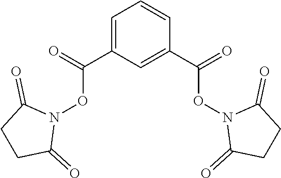 | 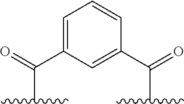 [SMILES: *C(=O)c1cccc(C(*)=O)c1] |
| 4 | 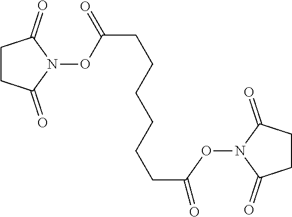 | 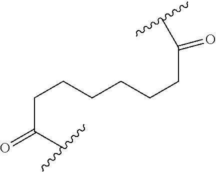 [SMILES: *C(=O)CCCCCCC(*)=O] |
| 5 | 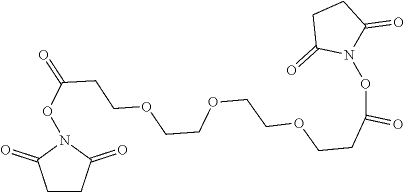 | 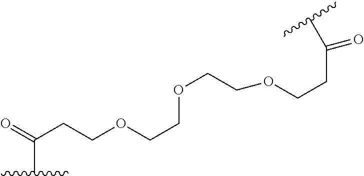 [SMILES: *C(=O)CCOCCOCCOCCC(*)=O] |
| 6 | 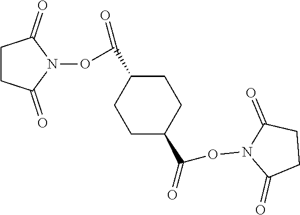 | 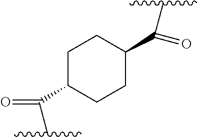 [SMILES: *C(=O)[C@H]1CC[C@H](C(*)=O)CC1] |
| 7 | 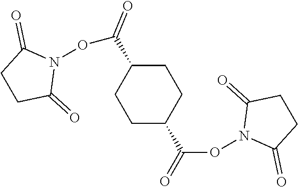 | 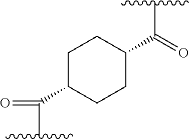 [SMILES: *C(=O)[C@H]1CC[C@@H](C(*)=O)CC1] |
| 8 | 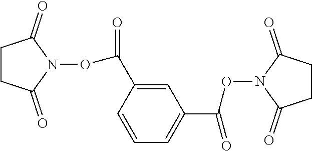 | 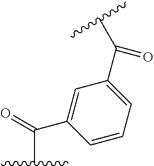 [SMILES: *C(=O)c1cccc(C(*)=O)c1] |
| 9 | 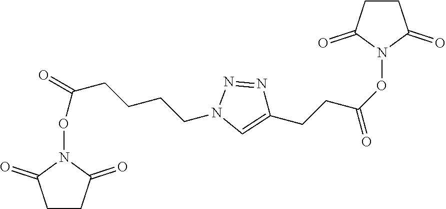 | 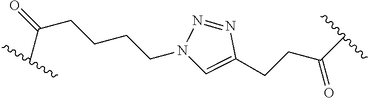 [SMILES: *C(=O)CCCCn1cc(CCC(*)=O)nn1] |
| 10 | 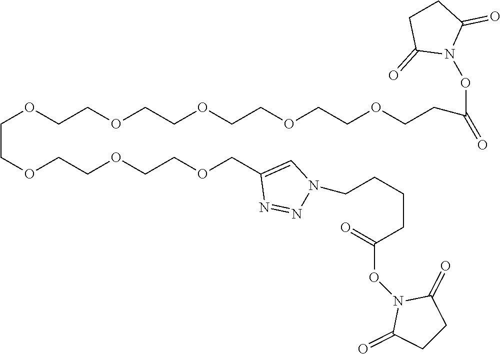 | 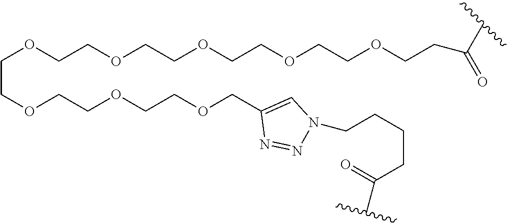 [SMILES: *C(=O)CCCCn1cc(COCCOCCOCCOCCOCCOCCOCCOCCC(*)=O)nn1] |
| 11 | 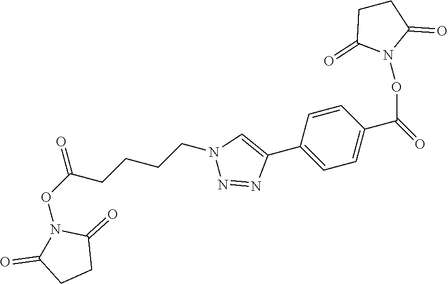 | 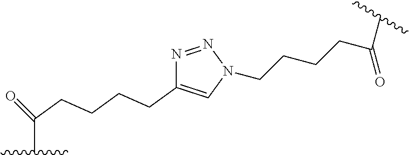 [SMILES: *C(=O)CCCCc1cn(CCCCC(*)=O)nn1] |
| 12 | 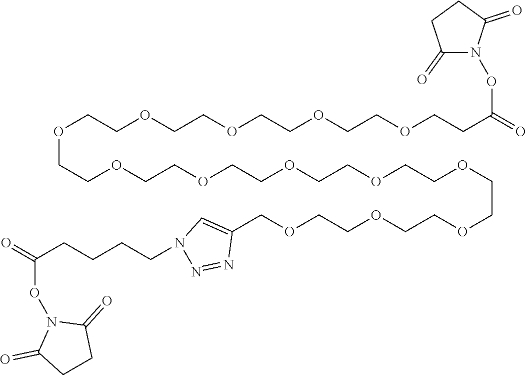 | 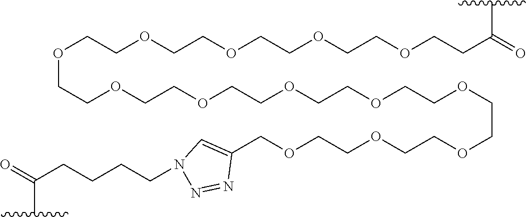 [SMILES: *C(=O)CCCCn1cc(COCCOCCOCCOCCOCCOCCOCCOCCOCCOCCOCCOCCOCCC(*)=O)nn1] |
| 13 | 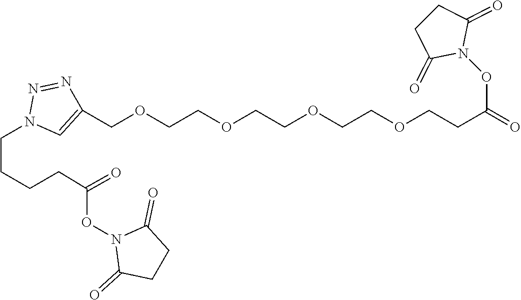 | 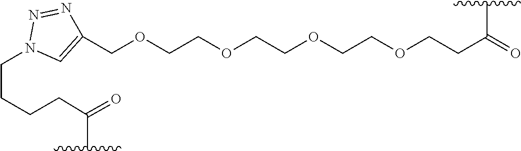 [SMILES: *C(=O)CCCCn1cc(COCCOCCOCCOCCC(*)=O)nn1] |
| 14 | 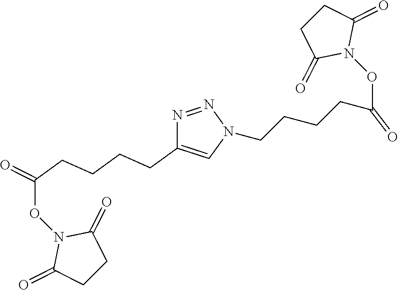 | 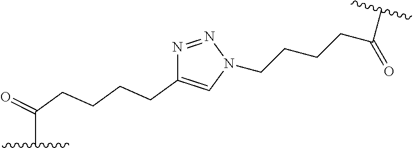 [SMILES: *C(=O)CCCCc1cn(CCCCC(*)=O)nn1] |
| 15 | 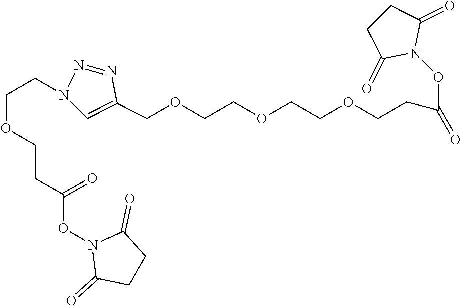 | 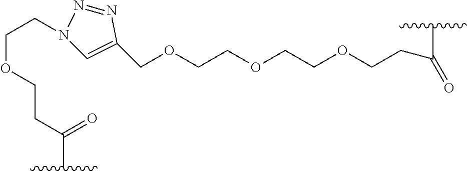 [SMILES: *C(=O)CCOCCOCCOCc1cn(CCOCCC(*)=O)nn1] |
| 16 | 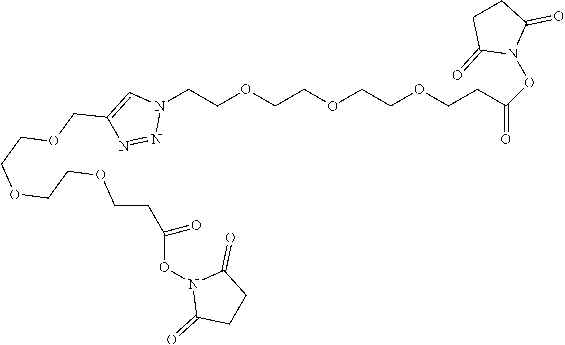 | 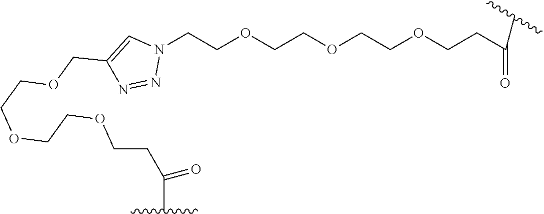 [SMILES: *C(=O)CCOCCOCCOCCn1cc(COCCOCCOCCC(*)=O)nn1] |
| 17 | 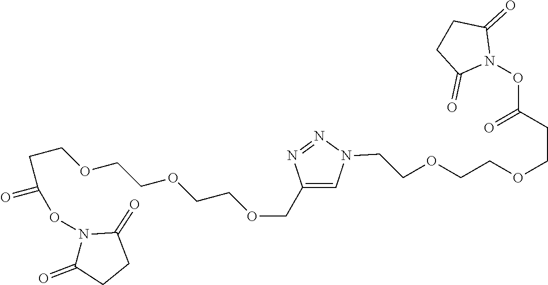 | 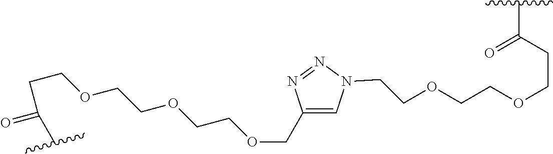 [SMILES: *C(=O)CCOCCOCCOCc1cn(CCOCCOCCC(*)=O)nn1] |
| 18 | 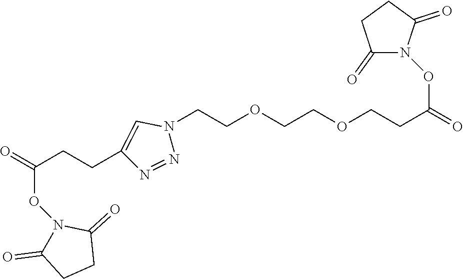 | 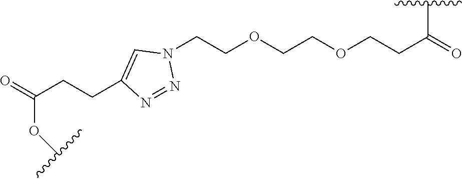 [SMILES: *OC(=O)CCc1cn(CCOCCOCCC(*)=O)nn1] |
| 19 | 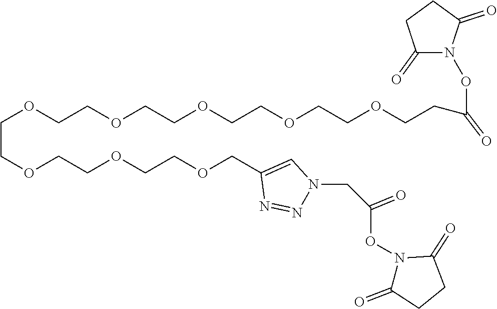 | 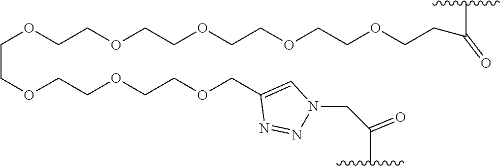 [SMILES: *C(=O)CCOCCOCCOCCOCCOCCOCCOCCOCc1cn(CC(*)=O)nn1] |
| 20 | 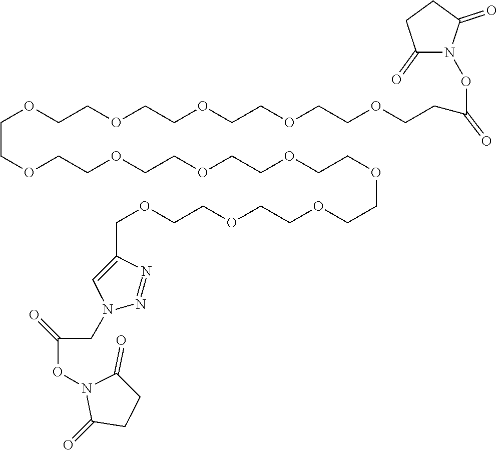 | 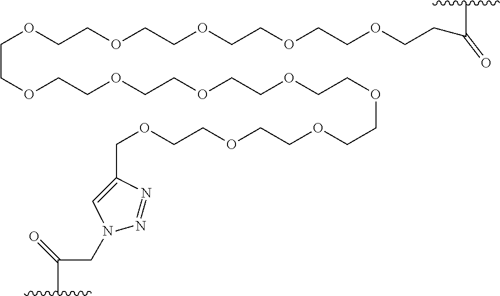 [SMILES: *C(=O)CCOCCOCCOCCOCCOCCOCCOCCOCCOCCOCCOCCOCCOCc1cn(CC(*)=O)nn1] |
| 21 | 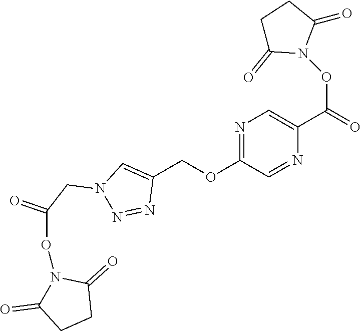 | 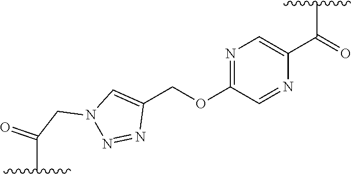 [SMILES: *C(=O)Cn1cc(COc2cnc(C(*)=O)cn2)nn1] |
| 22 | 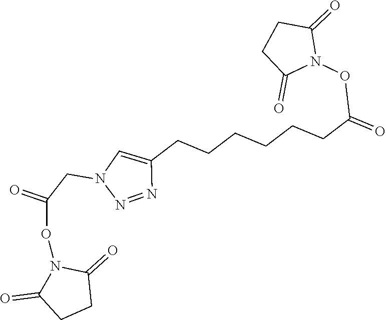 | 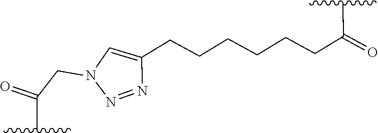 [SMILES: *C(=O)CCCCCCc1cn(CC(*)=O)nn1] |
| 23 | 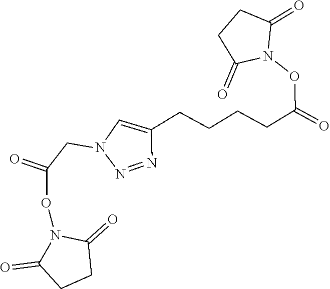 | 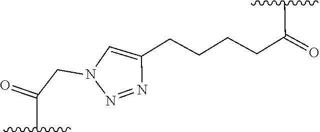 [SMILES: *C(=O)CCCCc1cn(CC(*)=O)nn1] |

Conjugation of a bifunctional linker to the epsilon amino group of the lysine residue at position B29 of the B-chain polypeptide of the first insulin or insulin analog molecule to the alpha amino group of the glycine residue at position A1′ of the A′-chain polypeptide of the second insulin or insulin analog molecules to form the insulin dimer linked by a linking moiety may be schematically shown as

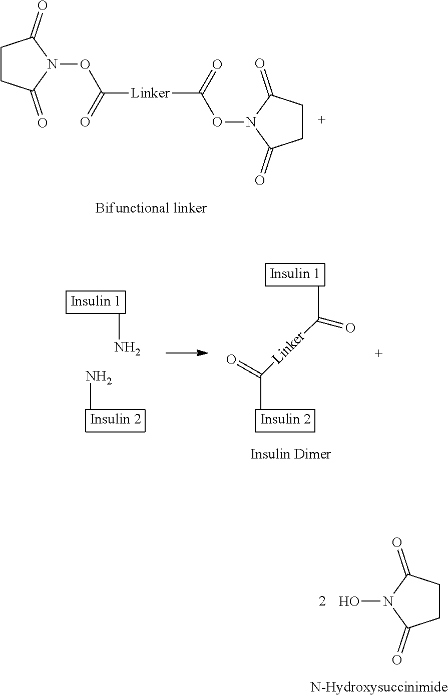

wherein the insulin 1 and insulin 2 molecules may be the same or different and the bifunctional linker and resulting linking moiety following conjugation may have the structure of any linker and resulting linking moiety disclosed herein.

### Modification of Insulin Polypeptides

In some embodiments, at least one of the A-chain polypeptides or B-chain polypeptides of the insulin receptor partial agonist is modified to comprise an acyl group. The acyl group can be covalently linked directly to an amino acid of the insulin polypeptide, or indirectly to an amino acid of the insulin polypeptide via a spacer, wherein the spacer is positioned between the amino acid of the insulin polypeptide and the acyl group. The insulin polypeptide may be acylated at the same amino acid position where a hydrophilic moiety is linked, or at a different amino acid position. For example, acylation may occur at any position including any amino acid of the A- or B-chain polypeptides as well as a position within the linking moiety, provided that the activity exhibited by the non-acylated insulin polypeptide is retained upon acylation. Non-limiting examples include acylation at positions A1 of the A chain and positions position B1 of the B chain.

In one specific aspect of the invention, the first and/or second insulin polypeptide (or derivative or conjugate thereof) is modified to comprise an acyl group by direct acylation of an amine, hydroxyl, or thiol of a side chain of an amino acid of the insulin polypeptide. In some embodiments, the first and/or second insulin polypeptide is directly acylated through the side chain amine, hydroxyl, or thiol of an amino acid. In this regard, an insulin polypeptide may be provided that has been modified by one or more amino acid substitutions in the A- or B-chain polypeptide sequence, including for example at positions B1, B10, or B22 or at any position of the linking moiety with an amino acid comprising a side chain amine, hydroxyl, or thiol. An example of a spacer as illustrated in Scheme X below,

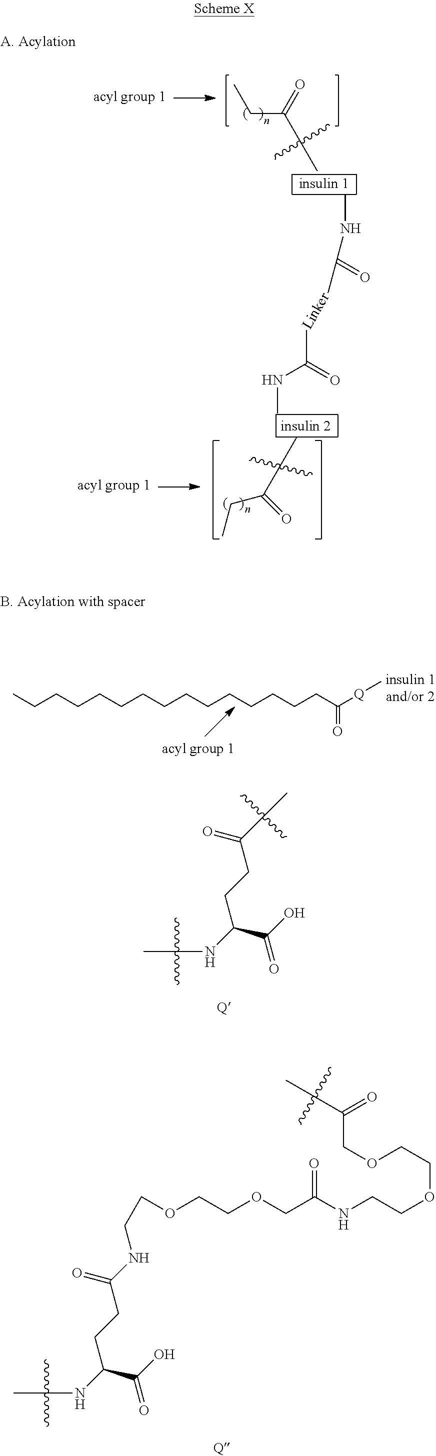

wherein in A. the wavy line illustrates point of attachment to side chain of insulin peptide and in B. the wavy line at the amino group of the spacer illustrates point of attachment to the acyl group 1 and the wavy line at the carbonyl group of the spacer illustrates point of attachment to the insulin peptide, Q is a spacer represented for example, as Q′ and Q″ and n is a C₁₅ alkyl chain

In some embodiments, the spacer between the first and/or second insulin polypeptide and the acyl group is an amino acid comprising a side chain amine, hydroxyl, or thiol (or a dipeptide or tripeptide comprising an amino acid comprising a side chain amine, hydroxyl, or thiol). In some embodiments, the spacer comprises a hydrophilic bifunctional spacer. In a specific embodiment, the spacer comprises an amino poly(alkyloxy)carboxylate. In this regard, the spacer can comprise, for example, NH₂(CH₂CH₂O)ₙ(CH₂)ₘCOOH, wherein m is any integer from I to 6 and n is any integer from 2 to 12, such as, e.g., 8-amino-3,6- dioxaoctanoic acid, which is commercially available from Peptides International, Inc. (Louisville, KY). In one embodiment, the hydrophilic bifunctional spacer comprises two or more reactive groups, e.g., an amine, a hydroxyl, a thiol, and a carboxyl group or any combinations thereof. In certain embodiments, the hydrophilic bifunctional spacer comprises a hydroxyl group and a carboxylate. In other embodiments. the hydrophilic bifunctional spacer comprises an amine group and a carboxylate. In other embodiments, the hydrophilic bifunctional spacer comprises a thiol group and a carboxylate.

In some embodiments. the spacer between the first and/or second insulin polypeptide and the acyl group is a hydrophobic bifunctional spacer. Hydrophobic bifunctional spacers are known in the art. See, e.g., Bioconjugate Techniques, G. T. Hermanson (Academic Press, San Diego, CA. 1996), which is incorporated by reference in its entirety. In certain embodiments. the hydrophobic bifunctional spacer comprises two or more reactive groups, e.g., an amine, a hydroxyl, a thiol, and a carboxyl group or any combinations thereof. In certain embodiments, the hydrophobic bifunctional spacer comprises a hydroxyl group and a carboxylate. In other embodiments, the hydrophobic bifunctional spacer comprises an amine group and a carboxylate. In other embodiments, the hydrophobic bifunctional spacer comprises a thiol group and a carboxylate. Suitable hydrophobic bifunctional spacers comprising a carboxylate and a hydroxyl group or a thiol group are known in the art and include, for example, 8-hydroxyoctanoic acid and 8-mercaptooctanoic acid.

In accordance with certain embodiments the bifunctional spacer can be a synthetic or naturally occurring amino acid comprising an amino acid backbone that is 3 to 10 atoms in length (e.g., 6-amino hexanoic acid, 5-aminovaleric acid, 7-aminoheptanoic acid, and 8-aminooctanoic acid). Alternatively, the spacer can be a dipeptide or tripeptide spacer having a peptide backbone that is 3 to 10 atoms (e.g., 6 to 10 atoms) in length. Each amino acid of the dipeptide or tripeptide spacer attached to the insulin polypeptide can be independently selected from the group consisting of: naturally-occurring and/or non-naturally occurring amino acids, including, for example, any of the D or L isomers of the naturally-occurring amino acids (Ala, Cys, Asp, Glu, Phe, Gly, His, Ile, Lys, Leu, Met, Asn, Pro, Arg, Ser, Thr, Val, Trp, Tyr), or any D or L isomers of the non-naturally occurring amino acids selected from the group consisting of: β-alanine (β-Ala), N-α-methyl-alanine (Me-Ala), aminobutyric acid (Abu), α-aminobutyric acid (γ-Abu), aminohexanoic acid (ε-Ahx), aminoisobutyric acid (Aib), aminomethylpyrrole carboxylic acid, aminopiperidinecarboxylic acid, aminoserine (Ams), aminotetrahydropyran-4-carboxylic acid, arginine N-methoxy-N-methyl amide, β-aspartic acid (β-Asp), azetidine carboxylic acid, 3-(2-benzothiazolyl)alanine, α-tert-butylglycine, 2-amino-5-ureido-n-valeric acid (citrulline, Cit), β-Cyclohexylalanine (Cha), acetamidomethyl-cysteine, diaminobutanoic acid (Dab), diaminopropionic acid (Dpr), dihydroxyphenylalanine (DOPA), dimethylthiazolidine (DMTA), γ-Glutamic acid (γ-Glu), homoserine (Hse), hydroxyproline (Hyp), isoleucine N-methoxy-N-methyl amide, methyl-isoleucine (MeIle), isonipecotic acid (Isn), methyl-leucine (MeLeu), methyl-lysine, dimethyl-lysine, trimethyl-lysine, methanoproline, methionine-sulfoxide (Met(O)), methionine-sulfone (Met(O2)), norleucine (Nle), methyl-norleucine (Me-Nle), norvaline (Nva), ornithine (Orn), para-aminobenzoic acid (PABA), penicillamine (Pen), methylphenylalanine (MePhe), 4-Chlorophenylalanine (Phe(4-Cl)), 4-fluorophenylalanine (Phe(4-F)), 4-nitrophenylalanine (Phe(4-NO2)), 4-cyanophenylalanine ((Phe(4-CN)), phenylglycine (Phg), piperidinylalanine, piperidinylglycine, 3,4-dehydroproline, pyrrolidinylalanine, sarcosine (Sar), selenocysteine (Sec), U-Benzyl-phosphoserine, 4-amino-3-hydroxy-6-methylheptanoic acid (Sta), 4-amino-5-cyclohexyl-3-hydroxypentanoic acid (ACHPA), 4-amino-3-hydroxy-5-phenylpentanoic acid (AHPPA), 1,2,3,4,-tetrahydro-isoquinoline-3-carboxylic acid (Tic), tetrahydropyranglycine, thienylalanine (Thi) , U-Benzyl-phosphotyrosine, O-Phosphotyrosine, methoxytyrosine, ethoxytyrosine, O-(bis-dimethylamino-phosphono)-tyrosine, tyrosine sulfate tetrabutylamine, methyl-valine (MeVal), 1-amino-1-cyclohexane carboxylic acid (Acx), aminovaleric acid, beta-cyclopropyl-alanine (Cpa), propargylglycine (Prg), allylglycine (Alg), 2-amino-2-cyclohexyl-propanoic acid (2-Cha), tertbutylglycine (Tbg), vinylglycine (Vg), 1-amino-1-cyclopropane carboxylic acid (Acp), 1-amino-1-cyclopentane carboxylic acid (Acpe), alkylated 3-mercaptopropionic acid, 1-amino-1-cyclobutane carboxylic acid (Acb). In some embodiments the dipeptide spacer is selected from the group consisting of: Ala-Ala, β-Ala-β-Ala, Leu-Leu, Pro-Pro, γ-aminobutyric acid-γ-aminobutyric acid, and γ-Glu-γ-Glu.

The first and/or second insulin polypeptide may be modified to comprise an acyl group by acylation of a long chain alkane. In specific aspects, the long chain alkane comprises an amine, hydroxyl, or thiol group (e.g. octadecylamine, tetradecanol, and hexadecanethiol) which reacts with a carboxyl group, or activated form thereof, of the insulin polypeptide. The carboxyl group, or activated form thereof, of the insulin polypeptide can be part of a side chain of an amino acid (e.g., glutamic acid, aspartic acid) of the insulin polypeptide or can be part of the peptide backbone.

In certain embodiments, the first and/or second insulin polypeptide is modified to comprise an acyl group by acylation of the long chain alkane by a spacer which is attached to the insulin polypeptide. In specific aspects, the long chain alkane comprises an amine, hydroxyl, or thiol group which reacts with a carboxyl group, or activated form thereof, of the spacer. Suitable spacers comprising a carboxyl group, or activated form thereof, are described herein and include, for example, bifunctional spacers, e.g., amino acids, dipeptides, tripeptides, hydrophilic bifunctional spacers and hydrophobic bifunctional spacers. As used herein, the term “activated form of a carboxyl group” refers to a carboxyl group with the general formula R(C═O)X, wherein X is a leaving group and R is the insulin polypeptide or the spacer. For example, activated forms of a carboxyl groups may include, but are not limited to, acyl chlorides, anhydrides, and esters. In some embodiments, the activated carboxyl group is an ester with an N-hydroxysuccinimide (NHS) leaving group.

With regard to these aspects of the invention, in which a long chain alkane is acylated by the peptide, the insulin polypeptide or the spacer, the long chain alkane may be of any size and can comprise any length of carbon chain. The long chain alkane can be linear or branched. In certain aspects, the long chain alkane is a C4 to C30 alkane. For example, the long chain alkane can be any of a C4 alkane, C6 alkane, C8 alkane, C10) alkane, C12 alkane, C14 alkane, C16 alkane, C18 alkane, C20 alkane, C22 alkane, C24 alkane, C26 alkane, C28 alkane, or a C30 alkane. In some embodiments, the long chain alkane comprises a C8 to C20 alkane, e.g., a C14 alkane, C16 alkane, or a C18 alkane.

In some embodiments, an amine, hydroxyl, or thiol group of the first and/or 10 second insulin polypeptide is acylated with a cholesterol acid. In a specific embodiment, the peptide is linked to the cholesterol acid through an alkylated des-amino Cys spacer, i.e., an alkylated 3-mercaptopropionic acid spacer. Suitable methods of peptide acylation via amines, hydroxyls, and thiols are known in the art. See, for example, Miller, Biochem Biophys Res Commun 218: 377-382 (1996); Shimohigashi and Stammer, Int J Pept Protein Res 19: 54-62 (1982); and Previero et al., Biochim Biophys Acta 263: 7-13 (1972) (for methods of acylating through a hydroxyl); and San and Silvius, J Pept Res 66: 169-180 (2005) (for methods of acylating through a thiol); Bioconjugate Chem. “Chemical Modifications of Proteins: History and Applications” pages 1, 2-12 (1990); Hashimoto et al., Pharmacuetical Res. “Synthesis of Palmitoyl Derivatives of Insulin and their Biological Activity” Vol. 6, No: 2 pp. 171-176 (1989).

The acyl group of the acylated peptide the first and/or second insulin polypeptide can be of any size, e.g., any length carbon chain, and can be linear or branched. In some specific embodiments of the invention, the acyl group is a C4 to C30 fatty acid. For example, the acyl group can be any of a C4 fatty acid, C6 fatty acid, C8 fatty acid, C10 fatty acid, C12 fatty acid, C14 fatty acid, C16 fatty acid, C13 fatty acid, C20 fatty acid, C22 fatty acid, C24 fatty acid, C26 fatty acid, C28 fatty acid, or a C30 fatty acid. In some embodiments, the acyl group is a C8 to C20 fatty acid, e.g., a C14 fatty acid or a C16 fatty acid. In some embodiments, the acyl group is urea. In an alternative embodiment, the acyl group is a bile acid. The bile acid can be any suitable bile acid, including, but not limited to, cholic acid, chenodeoxycholic acid, deoxycholic acid, lithocholic acid, taurocholic acid, glycocholic acid, and cholesterol acid.

The acylated first and/or second insulin polypeptide described herein can be further modified to comprise a hydrophilic moiety. In some specific embodiments the hydrophilic moiety can comprise a polyethylene glycol (PEG) chain. The incorporation of a hydrophilic moiety can be accomplished through any suitable means, such as any of the methods described herein. In some embodiments the acylated single chain analog comprises an amino acid selected from the group consisting of a Cys, Lys, Orn, homo-Cys, or Ac-Phe, and the side chain of the amino acid is covalently bonded to a hydrophilic moiety (e.g., PEG). In one embodiment, the acyl group is attached to position A1, A14, A15, B1, B2, B10, or B22 (according to the amino acid numbering of the A and B chains of native insulin), optionally via a spacer comprising Cys, Lys, Orn, homo-Cys, or Ac-Phe.

Alternatively, the acylated first and/or second insulin polypeptide comprises a spacer, wherein the spacer is both acylated and modified to comprise the hydrophilic moiety. Non-limiting examples of suitable spacers include a spacer comprising one or more amino acids selected from the group consisting of Cys, Lys, Orn, homo-Cys, and Ac-Phe.

In some embodiments, the amino terminus of at least one N-terminal amino acid of at least one of the A-chain polypeptides and the B-chain polypeptides of the insulin receptor partial agonist is modified to comprise a capping group. The capping group may be covalently linked directly to the amino group of the N-terminal amino acid or indirectly to the amino group via a spacer, wherein the spacer is positioned between the amino group of the N-terminal amino acid of the insulin polypeptide and the capping group. The capping group may be an acyl moiety as discussed supra. The capping group may have the general formula RC(O)—, where R can be R′CH2, R′NH, R′O, and R′ can be H (when R is R′CH₂ or R′NH), linear alkyl chain, amino acid, peptide, polyethylene glycol (PEG), saccharides, which in particular aspects RC(O)— may be acetyl, phenylacetyl, carbamoyl, N-alkyl carbamoyl, or alkoxycarbonyl. In particular aspects, the capping group is a carbamoyl group, acetyl group, glycyl, methyl group, methoxy acetyl group, dimethyl group, isobutyl group, PEG1 group, or PEG2 group (see Examples herein for structures of the capping groups). Carbamolyation of insulin has been disclosed by Oimoni et al., Nephron 46: 63-66 (1987) and insulin dimers comprising a carbamoyl groups at the N-terminus has been disclosed in disclosed in published PCT Application No. WO2014052451 (E.g., MIU-90).

In particular embodiments, at least one N-terminal amino acid is conjugated via the N2 nitrogen using a capping reagent comprising an N-hydroxysuccinimide ester linked to a group having the general formula RC(O). In particular embodiments, at least one N-terminal amino acid is conjugated via the N2 nitrogen to a capping group having the general formula RC(O)—, as defined supra. A subembodiment of this aspect of the invention is realized when aspects of RC(O) may be acetyl, phenylacetyl, carbamoyl, N-alkyl carbamoyl, or alkoxycarbonyl. In particular aspects, the capping group is a carbamoyl group, acetyl group, glycyl, methoxy acetyl group, dimethyl group, isobutyl group, PEG1 group, or PEG2 group. Still other particular aspects, the capping group is selected from Capping Group 1, 2, 3, 4, 5, 6, and 7.

Exemplary capping groups conjugated to the N-terminal amino group are illustrated in Table II.

TABLE II

| Capping | | |
|---|---|---|
| Group | | |
| No. | Capping Reagent | Capping Group |
| 1 | KNCO | 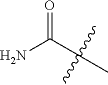 [SMILES: *C(N)=O] |
| 2 | 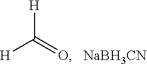 | (CH₃)₂ |
| 3 | 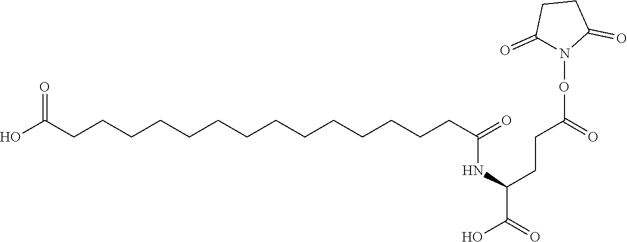 [SMILES: O=C(O)CCCCCCCCCCCCCCC(=O)N[C@@H](CCC(=O)ON1C(=O)CCC1=O)C(=O)O] | 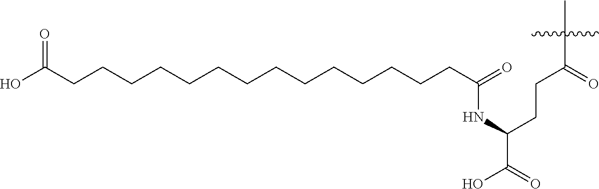 [SMILES: *C(=O)CC[C@H](NC(=O)CCCCCCCCCCCCCCC(=O)O)C(=O)O] |
| 4 | 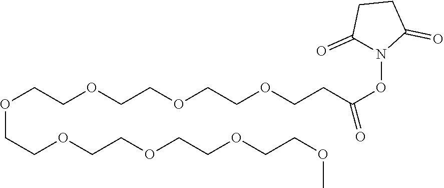 [SMILES: COCCOCCOCCOCCOCCOCCOCCOCCC(=O)ON1C(=O)CCC1=O] | 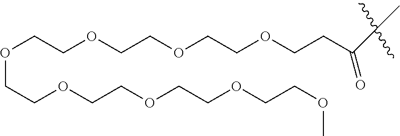 [SMILES: *C(=O)CCOCCOCCOCCOCCOCCOCCOCCOC] |
| 5 | 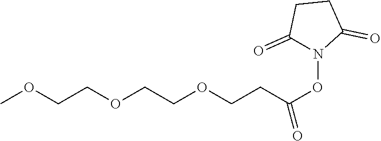 [SMILES: COCCOCCOCCC(=O)ON1C(=O)CCC1=O] | 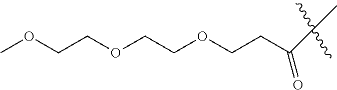 [SMILES: *C(=O)CCOCCOCCOC] |
| 6 | 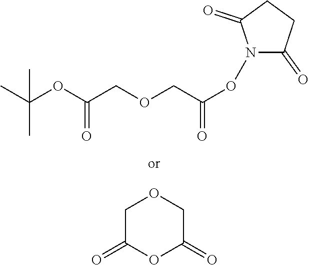 [SMILES: CC(C)(C)OC(=O)COCC(=O)ON1C(=O)CCC1=O ; O=C1COCC(=O)O1] | 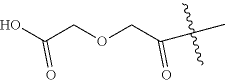 [SMILES: *C(=O)COCC(=O)O] |
| 7 | 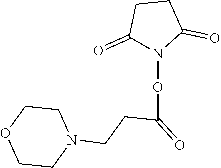 [SMILES: O=C(CCN1CCOCC1)ON1C(=O)CCC1=O] | 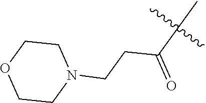 [SMILES: *C(=O)CCN1CCOCC1] |

wherein the wavy line indicates the bond between the capping group and the N-terminal amino group.

Thus, an embodiment of this invention is realized when the insulin dimer comprises a capping group conjugated to the N-terminal amino of at least one heterodimer B-chain is selected from the group consisting of acetyl, phenylacetyl, carbamoyl, N-alkyl carbamoyl, isobutyl, methoxy acetyl, 2-(carboxymethoxy)acetyl, glutaryl, Me2, carbamoyl, glycyl, AEG-C6, PEG1, PEG2, PEG8, and alkoxycarbonyl. A subembodiment of this aspect of the invention is realized when the capping group is selected from acetyl, phenylacetyl, carbamoyl, N-alkyl carbamoyl, isobutyl, methoxy acetyl, 2-(carboxymethoxy)acetyl, glutaryl, Me2, carbamoyl, Another subembodiment of this aspect of the invention is realized when the capping group is selected from acetyl, phenylacetyl, methoxy acetyl, 2-(carboxymethoxy)acetyl, Me2, and carbamoyl. Another subembodiment of this aspect of the invention is realized when the capping group is acetyl. Another subembodiment of this aspect of the invention is realized when the capping group is phenylacetyl. Another subembodiment of this aspect of the invention is realized when the capping group is methoxy acetyl. Another subembodiment of this aspect of the invention is realized when the capping group is 2-(carboxymethoxy)acetyl. Another subembodiment of this aspect of the invention is realized when the capping group is Me2. Another subembodiment of this aspect of the invention is realized when the capping group is carbamoyl.

Yet another aspect of the invention is realized when capping group is selected from the group consisting of acetyl, phenylacetyl, carbamoyl, N-alkyl carbamoyl, isobutyl, methoxy acetyl, 2-(carboxymethoxy)acetyl, glutaryl, Me2, carbamoyl, and the bifunctional linker moiety is selected from the group consisting of Linker 1, Linker 2, Linker 3, Linker 4, Linker 5, Linker 6, Linker 7, Linker 8, Linker 9, Linker 10, Linker 11, Linker 12, Linker 13, Linker 14, Linker 15, Linker 16, Linker 17, Linker 18, Linker 19, Linker 20, Linker 21, Linker 22, and Linker 23. Still another subembodiment of this aspect of the invention is realized when capping group is selected from the group consisting of acetyl, 2-(carboxymethoxy)acetyl, Me2, and carbamoyl and the bifunctional linker moiety is selected from the group consisting of Linker 1, Linker 2, Linker 3, Linker 4, Linker 5, Linker 6, Linker 7, Linker 8, Linker 9, Linker 10, Linker 11, Linker 12, Linker 13, Linker 14, Linker 15, Linker 16, Linker 17, Linker 18, Linker 19, Linker 20, Linker 21, Linker 22, and Linker 23.

### Exemplary Insulin Dimers

In particular embodiments, the present invention provides insulin dimers wherein a B29 Lys of a first insulin heterodimer molecule having a first A-chain polypeptide and first B-chain polypeptide and an A1′ Gly of a second insulin heterodimer having a second A′-chain polypeptide and second B′-chain polypeptide are conjugated together by a bifunctional linker selected from the group consisting Linker 1, Linker 2, Linker 3, Linker 4, Linker 5, Linker 6, Linker 7, Linker 8, Linker 9, Linker 10, Linker 11, Liner 12, Linker 13, Linker 14, Linker 15, Linker 16, Linker 17, Linker 18, Linker 19, Linker 20, Linker 21, Linker 22, and Linker 23.

In particular embodiments, at least one of the first A-chain or B-chain polypeptides is conjugated at its N-terminal amino acid to a capping group as disclosed herein or in some embodiments the N-terminal amino acids of the first insulin heterodimer molecule are conjugated to a capping group as disclosed. In particular embodiments, the capping group comprises the general formula RC(O)—, where R is discussed supra. A subembodiment of this aspect of the invention is realized when aspects of RC(O)— may be acetyl, phenylacetyl, methoxy acetyl, 2-(carboxymethoxy)acetyl, carbamoyl, N-alkyl carbamoyl, glycyl, AEG-C6, PEG1, PEG2, PEG8, or alkoxycarbonyl. Another subembodiment of this aspect of the invention is realized when the capping group is selected from Capping Group No. 1, 2, 3, 4, 5, 6, and 7.

A complete structural depiction of the compounds of this invention is illustrated with insulin Dimer 1 in Formula II below, wherein the B29 Lysine of one insulin heterodimer is conjugated to the A1′ Glycyl of the other insulin heterodimer through bis-functional C8 linker moiety; disulfide linkages between the Cys₆ and Cys₁₁ residues of the A-chain polypeptide (shown in Formula II) and disulfide linkages between the Cys₇ and Cys₂₀ of the A-chain to the Cys₇ and Cys₁₉ of the B-chain polypeptide, respectively exists; the linking moieties are covalently linking the epsilon-nitrogen of the lysine residue of B-chain with alpha-nitrogen of the terminal amino acid of A′-chain, wherein the A-chain and A′-chain polypeptide for Dimers 1-33 (Table III) has the amino acid sequence shown in SEQ ID NO:1; and the B-chain and B′-chain polypeptide for Dimers 1-33 has the amino acid sequence shown in SEQ ID NO:2, and where the capping group at the terminal nitrogen of A-chain and B-chain for insulin dimer 1 is carbamoyl, and the capping group for the epsilon-nitrogen of side chain of B29′ lysine is PEG8. It should be noted that the linking moiety and capping group in insulin dimer 1 independently may differ from other insulin dimers of the present invention as shown in Table III.

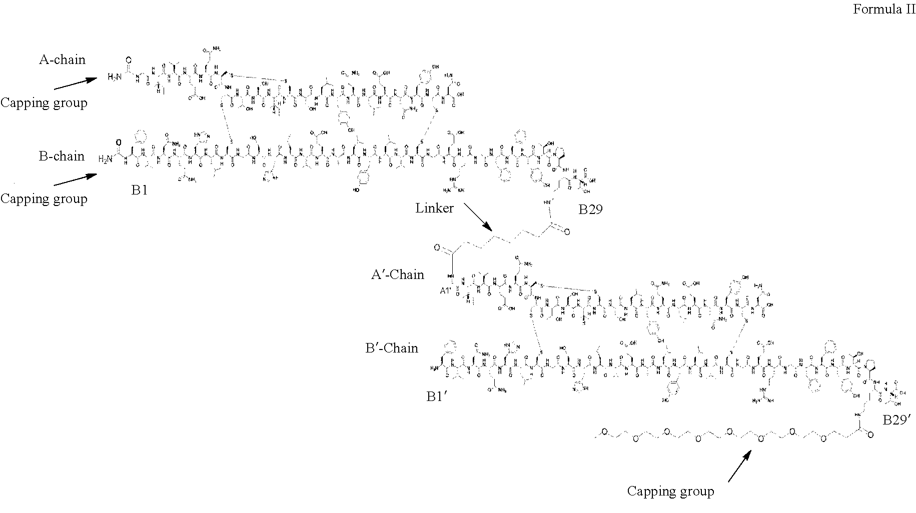

Exemplary insulin dimers include those in Table III:

TABLE III

| Dimer | Description |
|---|---|
| 1 | 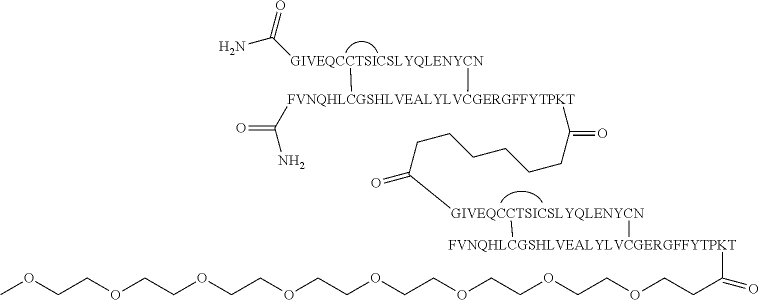 |
| 2 | 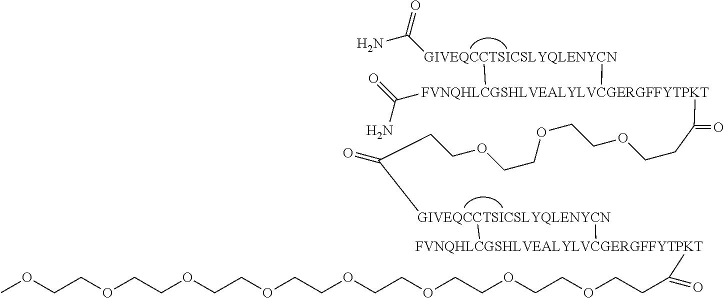 |
| 3 | 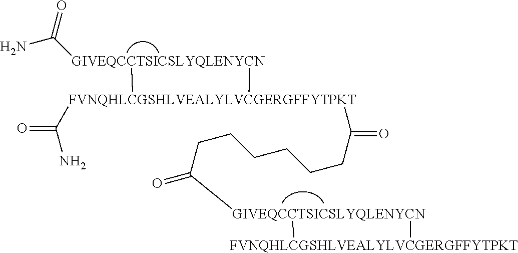 |
| 4 | 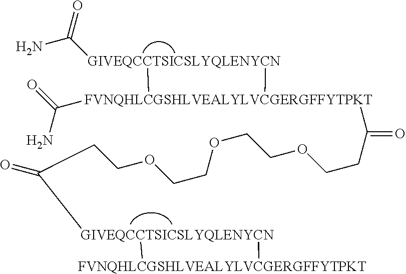 |
| 5 | 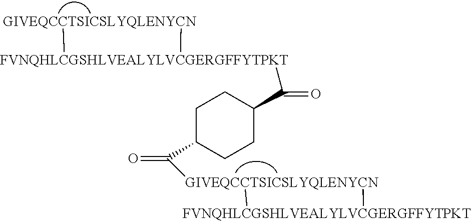 |
| 6 | 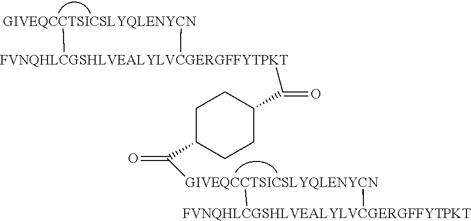 |
| 7 | 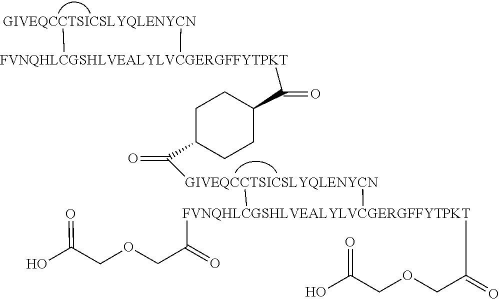 |
| 8 | 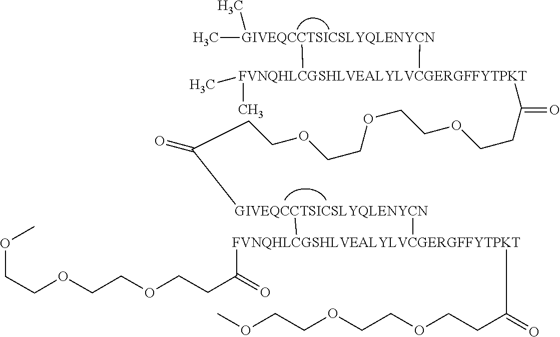 |
| 9 | 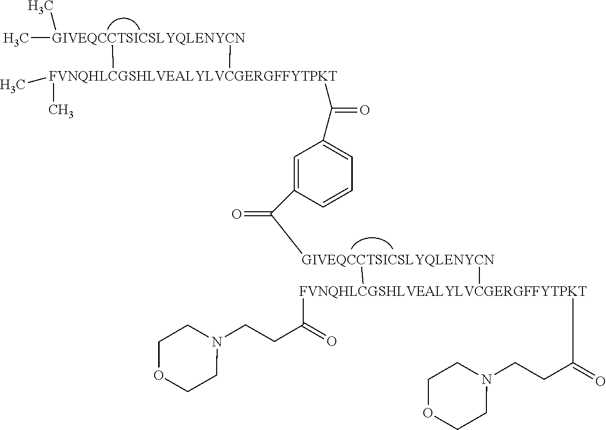 |
| 10 | 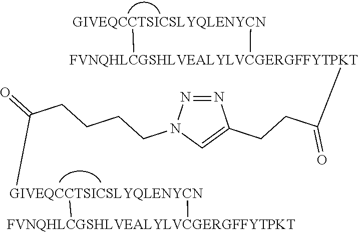 |
| 11 | 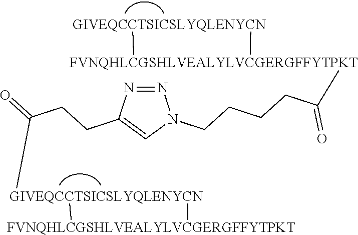 |
| 12 | 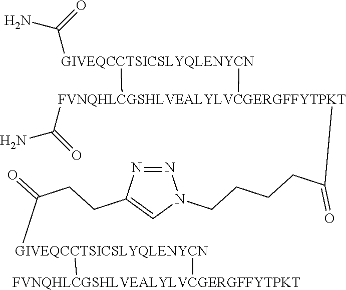 |
| 13 | 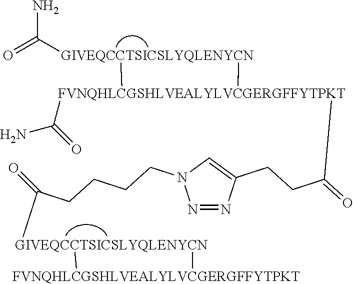 |
| 14 | 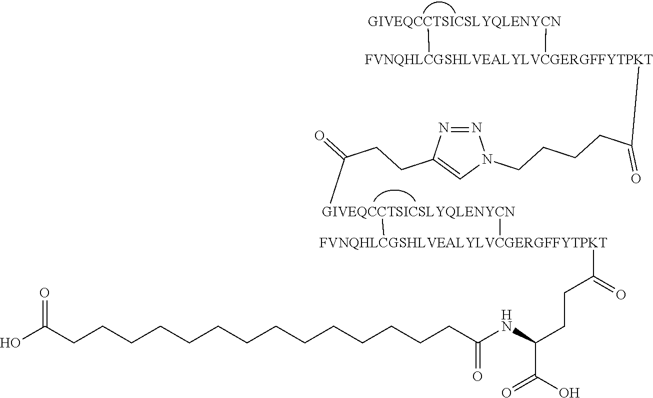 |
| 15 | 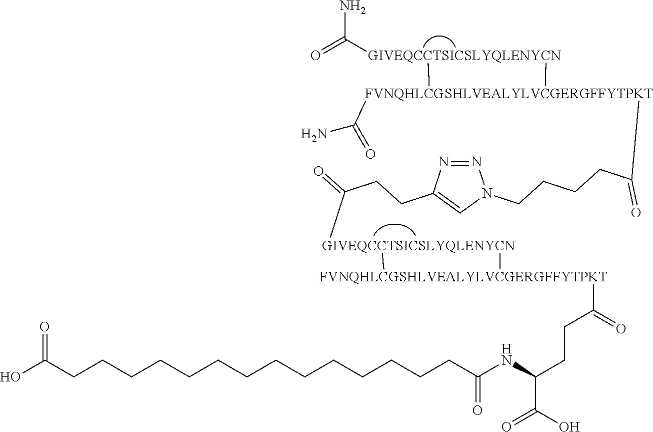 |
| 16 | 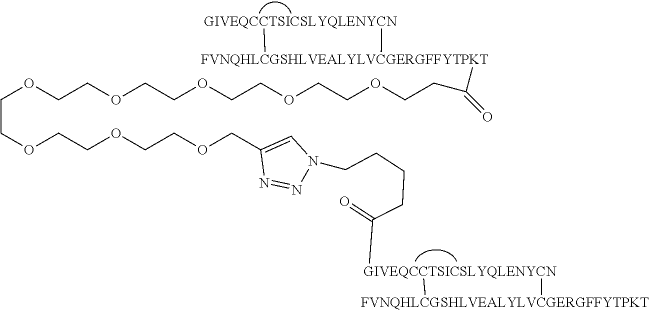 |
| 17 | 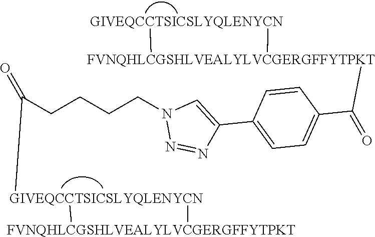 |
| 18 | 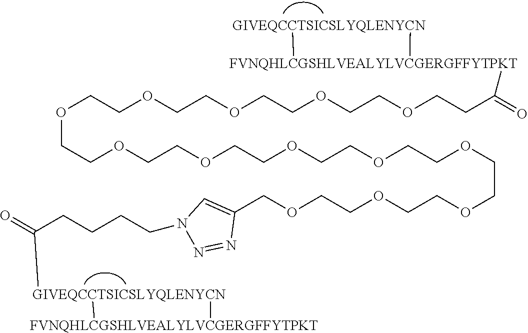 |
| 19 | 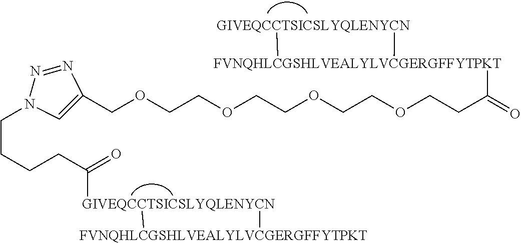 |
| 20 | 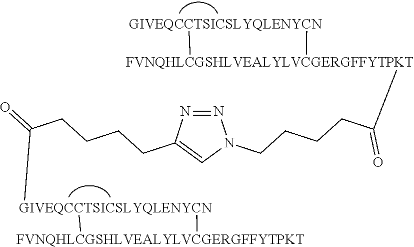 |
| 21 | 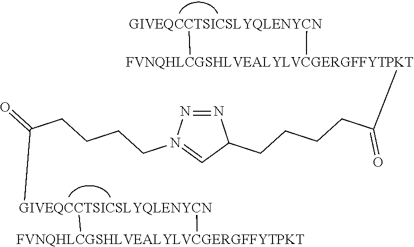 |
| 22 | 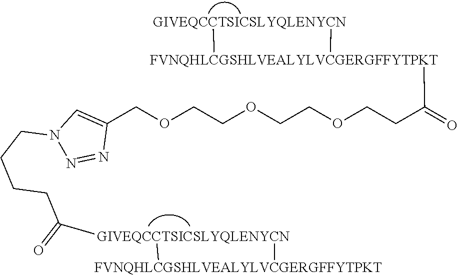 |
| 23 | 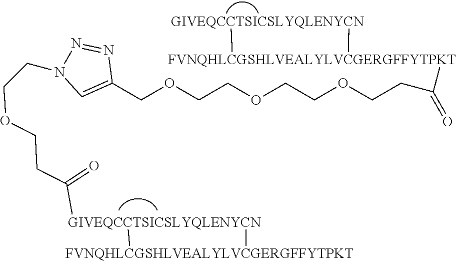 |
| 24 | 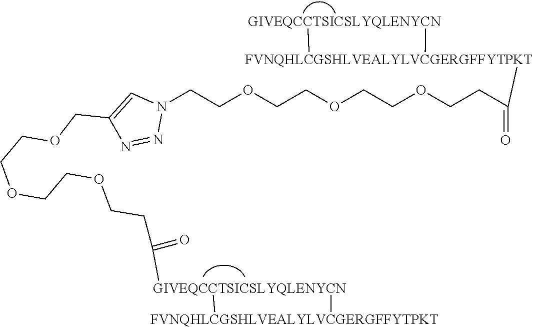 |
| 25 | 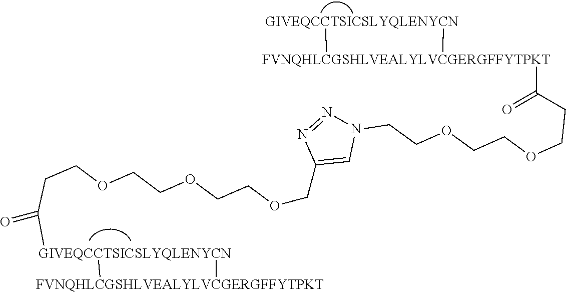 |
| 26 | 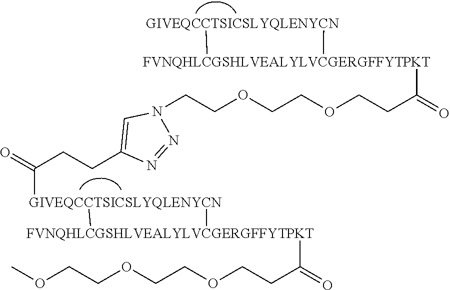 |
| 27 | 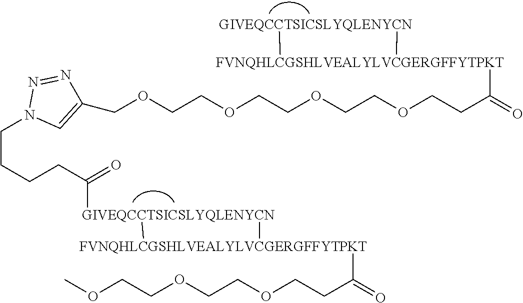 |
| 28 | 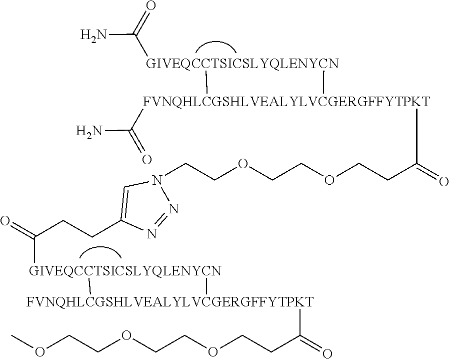 |
| 29 | 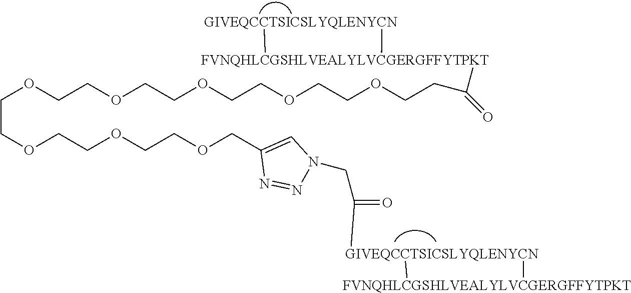 |
| 30 | 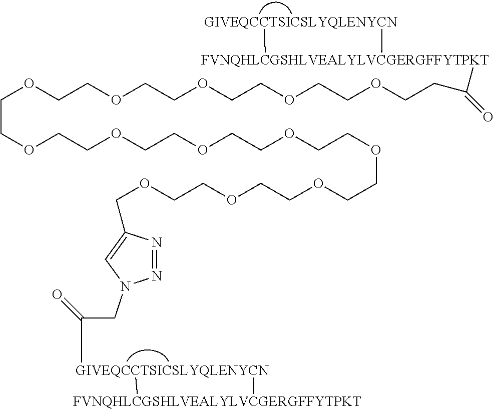 |
| 31 | 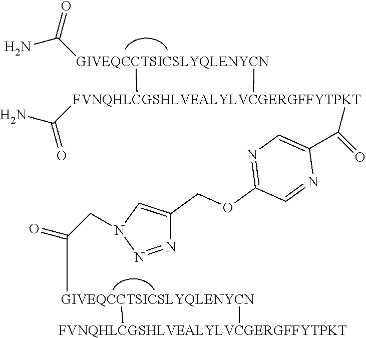 |
| 32 | 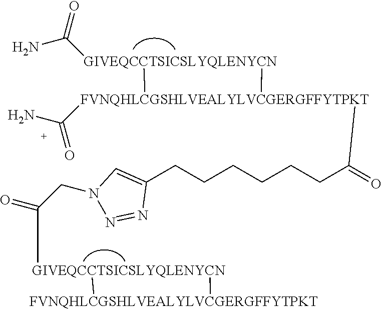 |
| 33 | 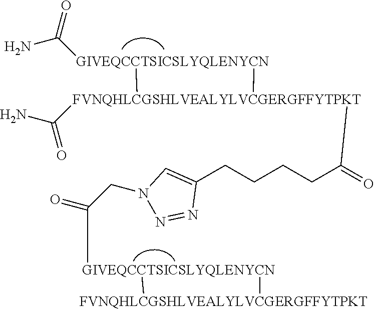 |

wherein the disulfide linkages between the Cys6 and Cys11 residues of the A-chain polypeptide and the disulfide linkages between the Cys7 and Cys20 of the A-chain to the Cys7 and Cys19 of the B-chain polypeptide, respectively, exists; linking the epsilon-nitrogen of the lysine residue of B-chain with alpha-nitrogen of the terminal amino acid of A′-chain, wherein the A-chain and A′-chain polypeptide for Dimers 1-33 has the amino acid sequence shown in SEQ ID NO: 1; and the B-chain and B′-chain for polypeptide Dimers 1-33 has the amino acid sequence shown in SEQ ID NO:2.

### Pharmaceutical Compositions

In accordance with one embodiment a pharmaceutical composition is provided comprising any of the novel insulin dimers disclosed herein, preferably at a purity level of at least 90%, 91%, 92%, 93%, 94%, 95%, 96%, 97%, 98% or 99%, and a pharmaceutically acceptable diluent, carrier or excipient. Such compositions may contain an insulin dimer as disclosed herein at a concentration of at least 0.5 mg/ml, 1 mg/ml, 2 mg/ml, 3 mg/ml, 4 mg/ml, 5 mg/ml, 6 mg/ml, 7 mg/ml, 8 mg/ml, 9 mg/ml, 10 mg/ml, 11 mg/ml, 12 mg/ml, 13 mg/ml, 14 mg/ml, 15 mg/ml, 16 mg/ml, 17 mg/ml, 18 mg/ml, 19 mg/ml, 20 mg/ml, 21 mg/ml, 22 mg/ml, 23 mg/ml, 24 mg/ml, 25 mg/ml or higher. In one embodiment the pharmaceutical compositions comprise aqueous solutions that are sterilized and optionally stored contained within various package containers. In other embodiments the pharmaceutical compositions comprise a lyophilized powder. The pharmaceutical compositions can be further packaged as part of a kit that includes a disposable device for administering the composition to a patient. The containers or kits may be labeled for storage at ambient room temperature or at refrigerated temperature.

The disclosed insulin dimers are believed to be suitable for any use that has previously been described for insulin peptides. Accordingly, the insulin dimers disclosed herein can be used to treat hyperglycemia, or treat other metabolic diseases that result from high blood glucose levels. Accordingly, the present invention encompasses pharmaceutical compositions comprising a insulin dimers as disclosed herein and a pharmaceutically acceptable carrier for use in treating a patient suffering from high blood glucose levels. In accordance with one embodiment the patient to be treated using an insulin dimer disclosed herein is a domesticated animal, and in another embodiment the patient to be treated is a human.

One method of treating hyperglycemia in accordance with the present disclosure comprises the steps of administering the presently disclosed insulin dimers to a patient using any standard route of administration, including parenterally, such as intravenously, intraperitoneally, subcutaneously or intramuscularly, intrathecally, transdermally, rectally, orally, nasally or by inhalation. In one embodiment the composition is administered subcutaneously or intramuscularly. In one embodiment, the composition is administered parenterally and the insulin polypeptide, or prodrug derivative thereof, is prepackaged in a syringe.

The insulin dimers disclosed herein may be administered alone or in combination with other anti-diabetic agents. Anti-diabetic agents known in the art or under investigation include native insulin, native glucagon and functional analogs thereof, sulfonylureas, such as tolbutamide (Orinase), acetohexamide (Dymelor), tolazamide (Tolinase), chlorpropamide (Diabinese), glipizide (Glucotrol), glyburide (Diabeta, Micronase, Glynase), glimepiride (Amaryl), or gliclazide (Diamicron); meglitinides, such as repaglinide (Prandin) or nateglinide (Starlix); biguanides such as metformin (Glucophage) or phenformin; thiazolidinediones such as rosiglitazone (Avandia), pioglitazone (Actos), or troglitazone (Rezulin), or other PPARγ inhibitors; alpha glucosidase inhibitors that inhibit carbohydrate digestion, such as miglitol (Glyset), acarbose (Precose/Glucobay); exenatide (Byetta) or pramlintide; Dipeptidyl peptidase-4 (DPP-4) inhibitors such as vildagliptin or sitagliptin; SGLT (sodium-dependent glucose transporter 1) inhibitors; or FBPase (fructose 1,6-bisphosphatase) inhibitors.

Pharmaceutical compositions comprising the insulin dimers disclosed herein can be formulated and administered to patients using standard pharmaceutically acceptable carriers and routes of administration known to those skilled in the art. Accordingly, the present disclosure also encompasses pharmaceutical compositions comprising one or more of the insulin dimers disclosed herein, or a pharmaceutically acceptable salt thereof, in combination with a pharmaceutically acceptable carrier. For example, the pharmaceutical compositions comprising the insulin dimers disclosed herein may optionally contain zinc ions, preservatives (e.g., phenol, cresol, parabens), isotonicizing agents (e.g., mannitol, sorbitol, lactose, dextrose, trehalose, sodium chloride, glycerol), buffer substances, salts, acids and alkalis and also further excipients. These substances can in each case be present individually or alternatively as mixtures. Glycerol, dextrose, lactose, sorbitol and mannitol are customarily present in the pharmaceutical preparation in a concentration of 100-250 mM, NaCl in a concentration of up to 150 mM. Buffer substances, such as, for example, phosphate, acetate, citrate, arginine, glycylglycine or TRIS (i.e. 2-amino-2-hydroxymethyl-1,3-propanediol) buffer and corresponding salts, are present in a concentration of 5-250 mM, commonly from about 10-100 mM. Further excipients can be, inter alia, salts or arginine.

In one embodiment the pharmaceutical composition comprises a 1 mg/mL concentration of the insulin dimer at a pH of about 4.0 to about 7.0 in a phosphate buffer system. The pharmaceutical compositions may comprise the insulin dimer as the sole pharmaceutically active component, or the insulin dimer can be combined with one or more additional active agents.

All therapeutic methods, pharmaceutical compositions, kits and other similar embodiments described herein contemplate that insulin dimers include all pharmaceutically acceptable salts thereof.

In one embodiment the kit is provided with a device for administering the insulin dimers composition to a patient. The kit may further include a variety of containers. e.g., vials, tubes, bottles, and the like. Preferably, the kits will also include instructions for use. In accordance with one embodiment the device of the kit is an aerosol dispensing device, wherein the composition is prepackaged within the aerosol device. In another embodiment the kit comprises a syringe and a needle, and in one embodiment the insulin dimer composition is prepackaged within the syringe.

The compounds of this invention may be prepared by standard synthetic methods, recombinant DNA techniques, or any other methods of preparing peptides and fusion proteins.

Although certain non-natural amino acids cannot be expressed by standard recombinant DNA techniques, techniques for their preparation are known in the art. Compounds of this invention that encompass non-peptide portions may be synthesized by standard organic chemistry reactions, in addition to standard peptide chemistry reactions when applicable.

The following examples are intended to promote a further understanding of the present invention.

### EXAMPLES

### General Procedures

All chemicals were purchased from commercial sources, unless otherwise noted. Reactions were usually carried out at room temperature unless otherwise noted. Reactions sensitive to moisture or air were performed under nitrogen or argon using anhydrous solvents and reagents. The progress of reactions was monitored by analytical thin layer chromatography (TLC), and ultra performance liquid chromatography-mass spectrometry (UPLC-MS). TLC was performed on E. Merck TLC plates precoated with silica gel 60F-254, layer thickness 0.25 mm. The plates were visualized using 254 nm UV and/or by exposure to cerium ammonium molybdate (CAM) orp-anisaldehyde staining solutions followed by charring. Ultra performance liquid chromatography (UPLC) was performed on a Waters Acquity™ UPLC® system.

UPLC-MS Method A: Waters Acquity™ UPLC® BEH C18 1.7 μm 1.0×50 mm column with gradient 10:90-95:5 v/v CH3CN/H2O+v 0.05% TFA over 2.0 min; flow rate 0.3 mL/min, UV wavelength 215 nm; UPLC-MS;

Method B: Waters Acquity™ UPLC® BEH C18 1.7 μm 2.1×100 mm column with gradient 20:80-90:10 v/v CH₃CN/H₂O+v 0.05% TFA over 4.0 min and 90:10-95:5 v/v CH₃CN/H₂O+v 0.05% TFA over 0.5 min; flow rate 0.3 mL/min, UV wavelength 200-300 nm; UPLC-MS;

Method C: Waters Acquity™ UPLC® BEH C18 1.7 μm 2.1×100 mm column with gradient 10:90-55:45 v/v CH₃CN/H₂O+v 0.05% TFA over 4.0 min and 55:45-95:5 v/v CH₃CN/H₂O+v 0.05% TFA over 40 sec; flow rate 0.3 mL/min, UV wavelength 200-300 nm; UPLC-MS;

Method D: Waters Acquity™ UPLC® BEH C8 1.7 μm 2.1×100 mm column with gradient 20:80-90:10 v/v CH₃CN/H₂O+v 0.1% TFA over 4.0 min and 90:10-95:5 v/v CH₃CN/H₂O+v 0.1% TFA over 0.4 min; flow rate 0.3 mL/min, UV wavelength 200-300 nm; Method E: Waters Acquity™ UPLC® BEH C8 1.7 μm 2.1×100 mm column with gradient 20:80-72.5:27.5 v/v CH3CNIH2O+v 0.05% TFA over 4.3 min and 72.5:27.5-95:5 v/v CH3CNIH2O+v 0.05% TFA over 0.5 min; flow rate 0.3 mL/min, UV wavelength 200-300 nm, and UPLC-MS;

Mass analysis was performed on a Waters SQ Detector with electrospray ionization in positive ion detection mode and the scan range of the mass-to-charge ratio was 170-900 or a Waters Micromass® LCT Premier™ XE with electrospray ionization in positive ion detection mode and the scan range of the mass-to-charge ratio was 300-2000. The identification of the produced insulin conjugates or IRPA was confirmed by comparing the theoretical molecular weight to the experimental value that was measured using UPLC-MS. For the determination of the linkage positions, specifically, insulin dimers were subjected to DTT treatment (for A/B chain) or Glu-C digestion (with or without reduction and alkylation), and then the resulting peptides were analyzed by LC-MS. Based on the measured masses, the linkage positions were deduced.

Flash chromatography was performed using either a Biotage Flash Chromatography apparatus (Dyax Corp.) or a CombiFlash® Rf instrument (Teledyne Isco). Normal-phase chromatography was carried out on silica gel (20-70 μm, 60 Å pore size) in pre-packed cartridges of the size noted. Ion exchange chromatography was carried out on a silica-based material with a bonded coating of a hydrophilic, anionic poly(2-sulfoethyl aspartamide) (PolySULFOETHYL A column, PolyLC Inc., 250×21 mm, 5 μm, 1000 Å pore size). Reverse-phase chromatography was carried out on C18-bonded silica gel (20-60 μm, 60-100 Å pore size) in pre-packed cartridges of the size noted. Preparative scale HPLC was performed on Gilson 333-334 binary system using Waters DELTA PAK C4 15 μm, 300 Å, 50×250 mm column or KROMASIL® C8 10 μm, 100 Å, 50×250 mm column, flow rate 85 mL/min, with gradient noted. Concentration of solutions was carried out on a rotary evaporator under reduced pressure or freeze-dried on a VirTis Freezemobile Freeze Dryer (SP Scientific).

Abbreviations: acetonitrile (AcCN), aqueous (aq), N,N-diisopropylethylamine or Hünig's base (DIPEA), dichlormethane (DCM), N,N-dimethylformamide (DMF), dimethyl sulfoxide (DMSO), ethyl acetate (EtOAc), N-(3-dimethylaminopropyl)-N′-ethylcarbodiimide hydrochloride (EDC), gram(s) (g), 1-hydroxybenzotriazole hydrate (HOBt), hour(s) (h or hr), hydrochloride (HCl), mass spectrum (ms or MS), microgram(s) (μg), microliter(s) (μL), micromole (μmol), milligram(s) (mg), milliliter(s) (mL), millimole (mmol), minute(s) (min), retention time (Rₜ), room temperature (rt), saturated (sat. or sat'd), saturated aq sodium chloride solution (brine), sodium hydroxide (NaOH), tris[(1-benzyl-1H-1,2,3-triazol-4-yl)methyl]amine, tangential flow filtration (TFF), triethylamine (TEA), trifluoroacetic acid (TFA), and N,N,N′,N′-tetramethyl-O-(N-succinimidyl)uronium tetrafluoroborate (TSTU), isopropyl acetate (IPAC), methyl tert-butyl ether (MTBE), 1,1,3,3-Tetramethylguanidine (TMG), 2,2,6,6-Tetramethylpiperidine (TMP); molecular weight cut off (MWCO).

The term “RHI” refers to recombinant human insulin and is used to indicate that the insulin has the amino acid sequence characteristic of native, wild-type human insulin. As used herein in the tables, the term indicates that the amino acid sequence of the insulin comprising the dimer is that of native, wild-type human insulin.

### PREPARATIVE EXAMPLES

### Preparative Example 1

### Bis(2,5-dioxopyrrolidin-1-yl) (1S,4S)-cyclohexane-1,4-dicarboxylate (trans-cyclohexane 1,4-diacid) (Linker 1)

To a solution of trans-cyclohexane-1,4-dicarboxylic acid (200 mg, 1.162 mmol) in DCM (11 mL) at 0° C. was added TSTU (734 mg, 2.44 mmol) and DIPEA (0.5 mL, 2.86 mmol). The resulting reaction mixture was stirred at room temperature for 1 hour. The product was crushed out in reaction solution as a solid; filtered and washed with DCM (2×5 mL); and dried in vacuo to obtain the title compound. UPLC-MS Method B: Rt=3.20 min, m/z=367.16 [M+1]. ¹H NMR (500 MHZ, DMSO): δ 2.81-2.89 (m; 2H); 2.80 (s; 8H); 2.02-2.10 (m; 4H); 1.57-1.63 (m; 4H).

### Preparative Example 2

### Bis(2,5-dioxopyrrolidin-1-yl) (1R,4R)-cyclohexane-1,4-dicarboxylate (Linker 2)

To a solution of (1R,4R)-cyclohexane-1,4-dicarboxylic acid (200 mg, 1.162 mmol) in DCM (11 mL) at 0° C. was added TSTU (734 mg, 2.439 mmol) and DIPEA (0.5 mL, 2.86 mmol). The resulting reaction mixture was stirred at room temperature for 1 hour. The residue was purified by silica gel chromatography (0-100% EtOAc/Hexanes) to provide the title compound. UPLC-MS Method B: Rt=3.17 min, m/z=366.11 [M+1]. ¹H NMR (500 MHz, DMSO): δ 3.02-3.08 (m; 2H); 2.80 (s; 8H); 1.80-1.90 (m; 8H).

### Preparative Example 3

### Bis(2,5-dioxopyrrolidin-1-yl) isophthalate (bis(2,5-dioxopyrrolidin-1-yl) isophthalate (Linker 3)

Using isophthalic acid, the title product was prepared in similar fashion to Linker 1. UPLC-MS Method A: Rt=0.73 min, m/z=721.21 [2M+1].

### Preparative Example 4

### N^{2,1A},N^{2,1B}-bis(carbamoyl) Human Insulin (Analog 1)

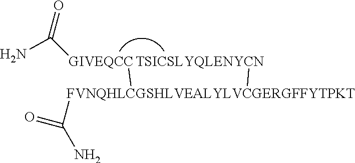

To a suspension of RHI (1.0 g, 0.172 mmol) in water (50 mL) was added a solution of potassium phosphate, dibasic (0.249 g, 1.429 mmol) in water (5.0 mL). After stirring at room temperature for 30 minutes, to the resulting mixture was added potassium cyanate (0.279 g, 3.44 mmol). The reaction mixture was allowed to stir for 16 hours. To stop the reaction, unreacted potassium cyanate was removed by TFF using MWCO 3K diafiltration device, and the product was isolated as a solid by lyophilization. The product contained about 10-35% of A1/B1/B29-tris-urea-RHI, which optionally could be removed by reverse-phase chromatography on C8 phase (KROMASIL C8 250×50 mm, 10 μm, 100 Å column; Buffer A: 0.05% TFA in deionized water; Buffer B: 0.05% TFA in AcCN), flow rate=85 mL/min, gradient B in A 26-34% over 30 min. UPLC-MS Method C: Rt=4.29 min, m/z=1474.6 [(M+4/4)]. The N-terminal capping has the structure

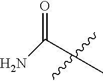 [SMILES: *C(N)=O]

(carbamoyl) wherein the wavy line indicates the bond between the capping group and the N2 nitrogen of the N-terminal amino acid.

### Preparative Example 5

### N^{2,1A},N^{2,1B}[tetrakis(methyl)] Human Insulin (Analog 2)

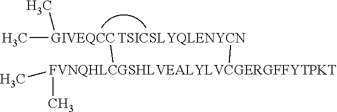

A suspension of RHI (200 mg, 0.034 mmol) in water (10 ml) and DMSO (1.0 mL) was acidified to pH=2.5 with acetic acid until a clear solution was obtained. The pH was readjusted with 1M NaOH to pH=4.0 and 19.3 μL of 37% formaldehyde/water solution was added. A solution of sodium cyanoborohydride (2.164 mg, 0.034 mmol) in water (1.0 mL) was added and stirred for 1 hr. The reaction was quenched with ethanolamine (100 μl, 1.653 mmol) and the product was isolated by reverse-phase chromatography (KROMASIL C8 250×50 mm, 10 μm, 100 Å column; Buffer A: 0.05% TFA in deionized water; Buffer B: 0.05% TFA in AcCN, flow rate 85 mL/min, gradient B in A 26-33% in 30 min). UPLC-MS Method C: Rt=3.56 min, m/z=1466.80 [(M+4)/4].

The N-terminal capping group has the structure

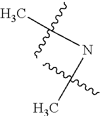 [SMILES: *N*]

wherein the wavy line indicates the bond between the methyl capping groups and the N² nitrogen of the N-terminal amino acid.

### Preparative Example 6

### General Method A. Synthesis of N^{6,29B} Acylated Human Insulins (Analogs)

In an appropriately sized container, insulin or insulin analog was dissolved, with gentle stirring, at room temperature in a mixed solvent: 2:3 v/v 0.1 M Na₂CO₃:AcCN. After the mixture cleared, the pH was adjusted to the value of 10.5-10.8 using alkaline solution, e.g., 0.1 N NaOH. In a separate vial, an activated ester intermediate (a Capping Reagent) was dissolved in an organic solvent, e.g., DMSO, at room temperature. Aliquots of the solution of the activated ester (a Capping Reagent) were added over a period of time to the solution containing insulin until UPLC-MS chromatogram showed that most of the unmodified insulin had been reacted and that a substantial portion of the reaction mixture had been converted into B29-conjugated insulin. The reaction was quenched by the addition of an amine nucleophile, e.g., 2-aminoethanol. The reaction solution was stirred at room temperature for 30 minutes. The resulting solution was carefully diluted with cold H₂O (20×) at 0° C. and its pH was adjusted to a final pH of 2.5 using 1 N HCl. The solution was first concentrated by ultrafiltration, either through a tangential flow filtration (TFF) system or using Amicon Ultra-15 Centrifugal Units, with 1K, 3K or 10K MWCO membrane. The resulting solution was then further purified by reverse phase HPLC (Kromasil C8 250×50 mm, 10 μm, 100 Å column; Buffer A: 0.05-0.1% TFA in water; Buffer B: 0.05-0.1% TFA in AcCN). Fractions containing the title conjugate were combined and freeze-dried or buffer exchanged using TFF system and/or Amicon Ultra-15 to give the title product. Optionally, the material can also be subjected to ion exchange chromatography (PolySULFOETHYL A column, PolyLC Inc., 250×21 mm, 5 □mμm, 1000 Å; Buffer A: 0.1% (v/v) H₃PO₄/25% AcCN; Buffer B: 0.1% (v/v) H₃PO₄/25% AcCN/0.5 M NaCl). Fractions containing B29-conjugate with desired purity are combined, concentrated using TFF system or Amicon Ultra-15, and de-salted by reverse phase HPLC as described earlier.

As an alternative to the acylation in aqueous media with Na₂CO₃ as the base described above, RHI may be dissolved in an a polar organic solvent (e.g. DMSO) and treated with 20-40 eq. of a strong organic base, such as TMG or TMP followed by a dropwise addition of a solution of the acylating agent in DMSO. The resulting mixture is stirred for 20-40 min, and then added dropwise to 50-100 vol. of a stirred mixture of 5:1 IPAC:MTBE. After stirring for 15 minutes, the suspended solids are collected via filtration. washed with 5:1 IPAC:MTBE, and the cake is dried. The product is purified as described above.

### Preparative Example 7

### N^{6,29B}(2,5,8,11,14,17,20,23-octaoxahexacosan-26-oyl) Human Insulin (Analog 3)

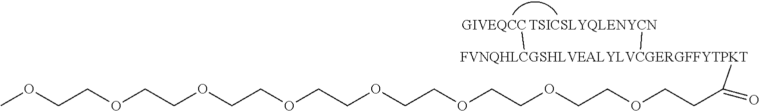

Using the procedures of General Method A and 2,5-dioxopyrrolidin-1-yl 2,5,8,11,14,17,20,23-octaoxahexacosan-26-oate as the capping agent, the title compound was isolated as a solid after reverse-phase chromatography on C8 phase (Column KROMASIL, C8 10 μm 100 Å, 250×50 mm; solvent A=water/0.05% TFA, solvent B=AcCN/0.05% TFA), flow rate=85 mL/min, gradient B in A 26-43% over 30 min). UPLC-MS Method C, Rt=3.64 min, m/z=1551.29 [(M+4)/4].

### Preparative Example 8

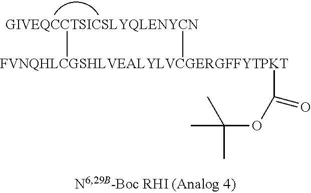

RHI (20 g, 3.44 mmol) was dissolved in DMSO (180 mL) and 1,1,3,3-tetramethylguanidine (8.64 mL, 68.9 mmol) was added followed by a dropwise addition of a solution of tert-butyl (2,5-dioxopyrrolidin-1-yl) carbonate (0.741 g, 3.44 mmol) in DMSO (20 mL). The resulting mixture was stirred for 40 min, and was then added dropwise to a stirred mixture of 5:1 IPAC:MTBE (2000 mL). After stirring for 15 minutes, the suspended solid was collected via filtration and washed with 3×200 mL 5:1 IPAC:MTBE. The cake was dried by pulling vacuum through it with a stream of nitrogen above it overnight. The desired material was purified by HPLC (KROMASIL C8 250×50 mm, 10 μm, 100 Å column; Buffer A: 0.05-0.1% TFA in deionized water; Buffer B: 0.05-0.1% TFA in AcCN). UPLC-MS Method D: Rt=2.88 min, m/z=1477.54 [(M+4)/4].

### Preparative Example 9

### N^{6,29B}-(pent-4-ynoyl) Human Insulin (Analog 5)

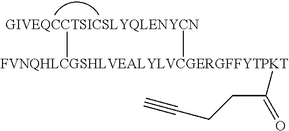

Using 2,5-dioxopyrrolidin-1-yl pent-4-ynoate as the reagent and General Method A, the title material was obtained. UPLC-MS Method C, Rt=4.08 min, m/z=1472.56 [(M+4)/4].

### Preparative Example 10

### General Method B: Synthesis of Insulins (Analogs) with Capping Groups at Both B1 and B29 Positions is Described

To a solution A1-TFA-RHI (F. Liu et. al., Journal of Peptide Sci., 2012, 18, 336-341) in DMSO (optionally generated in situ from RHI and ethyl trifluoroacetate) was added a DMSO solution of an activated ester intermediate (Capping Reagent) is added at room temperature. The mixture was stirred until UPLC-MS chromatogram showed that most of the A1-TFA-RHI has been reacted and that a substantial portion of the reaction mixture had been converted into A1-TFA, B1,B29-capped insulin. The reaction mixture was then mixed with commercial concentrated ammonium hydroxide solution. Optionally, before treatment with ammonium hydroxide, the DMSO solvent of the reaction mixture can be removed either by exchange for water using a tangential flow filtration (TFF) system or using Amicon Ultra-15 Centrifugal Units, with 1K, 3K or 10K MWCO membrane, or by precipitation of the product by addition of the reaction mixture into 50-100 volumes of weak organic solvent, such as ether, MTBE, IPAC, or a mixture of thereof. After confirmation by UPLC-MS that most of A1-TFA group had been cleaved by ammonium hydroxide, the solution was diluted with water and neutralized by addition of 3N HCl to pH=7.5. The solution was concentrated using a tangential flow filtration (TFF) system or using Amicon Ultra-15 Centrifugal Units, with 1K, 3K or 10K MWCO membrane, and the product was purified by reverse phase HPLC (Kromasil C8 250×50 mm, 10 μm, 100 Å column; Buffer A: 0.05% TFA in water; Buffer B: 0.05% TFA in AcN). Fractions containing the title conjugate were combined and freeze-dried or buffer exchanged using TFF system and/or Amicon Ultra-15 to give insulin acylated with a capping group at B1 and B29 sites.

### Preparative Example 11 (Analog 6)

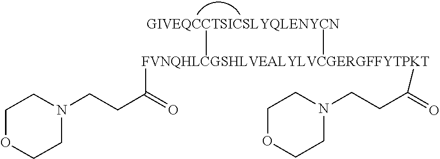

Using 2,5-dioxopyrrolidin-1-yl 3-(2-(2-methoxyethoxy)ethoxy)propanoate as the capping reagent, the title material was synthesized using procedures of General Method B. The product was purified by reverse-phase chromatography (KROMASIL C8 250×50 mm, 10 μm, 100 Å column; Buffer A: 0.05-0.1% TFA in deionized water; Buffer B: 0.05-0.1% TFA in AcCN, B in A 29-35% in 30 min.). UPLC-MS Method C, Rt=4.08 min, m/z=1523.41 [(M+4)/4].

### Preparative Example 12

### N^{2,1B},N^{6,29B}-bis(3-(2-(2-methoxyethoxy)ethoxy)propanoyl) Human Insulin (Analog 7)

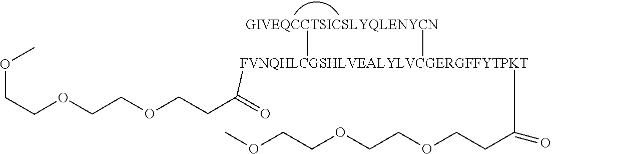

Starting from A1-TFA-RHI (F. Liu et. al., Journal of Peptide Sci., 2012, 18, 336-341), using 2,5-dioxopyrrolidin-1-yl 3-(2-(2-methoxyethoxy)ethoxy)propanoate as the capping reagent and procedures of General method B, the title compound was obtained as a solid after reverse-phase chromatography on C8 phase (Column KROMASIL, C8 10 μm 100 Å, 250×50 mm; solvent A=water/0.05% TFA, solvent B=AcCN/0.05% TFA), flow rate=85 mL/min, gradient B in A 26-33% over 30 min). UPLC-MS Method C, Rt=4.18 min, m/z=1939.67 [(M+4)/4].

### Preparative Example 13

### General Method C. N^{2,1A} Insulin Conjugates (Analogs)

In an appropriately sized container, insulin was dissolved, with gentle stirring, at rt in an aqueous pH=3.0 solvent. After the mixture cleared, the pH was adjusted to the value of 8.0-8.5 using alkaline solution, e.g., 0.1 N NaOH. In a separate vial, an activated ester intermediate was dissolved in an organic solvent, e.g., DMSO, at rt. Aliquots of the solution of the activated ester was added over a period of time to the solution containing insulin until UPLC-MS chromatogram showed that most of the unmodified insulin had been reacted and that a substantial portion of the reaction mixture had been converted into A1-conjugated insulin. The reaction was quenched by the addition of an amine nucleophile, e.g., 2-aminoethanol. The reaction solution was stirred at rt for 30 min. The resulting solution was carefully diluted with cold H₂O (20×) at 0° C. and its pH was adjusted to a final pH of 2.5 using 1 N HCl (and 0.1 N NaOH if needed). The solution was first concentrated by ultrafiltration, either through a tangential flow filtration (TFF) system or using Amicorn Ultra-15 Centrifugal Units, with 1K, 3K or 10K MWCO membrane. The concentrated solution was usually first subjected to ion exchange chromatography (PolySULFOETHYL A column, PolyLC Inc., 250×21 mm, 5 μm, 1000 Å; Buffer A: 0.1% (v/v) H₃PO₄/25% AcCN; Buffer B: 0.1% (v/v) H₃PO₄/25% AcCN/0.5 M NaCl). Fractions containing B29-conjugate with desired purity were combined and concentrated using TFF system or Amicon Ultra-15. The resulting solution was then further purified by reverse phase HPLC (Waters C4 250×50 mm column, 10 μm, 1000 Å column or Kromasil C8 250×50 mm, 10 μm, 100 Å column; Buffer A: 0.05-0.1% TFA in water; Buffer B: 0.05-0.1% TFA in AcCN). Fractions containing the title conjugate were combined and freeze-dried or buffer exchaged using TFF system and/or Amicorn Ultra-15 to give the title product.

### Preparative Example 14 (Analog 8)

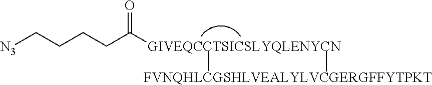

The title compound was synthesized according to General Method C. UPLC-MS Method C: Rt=3.04 min, m/z=1487.855 [(M+4)/4].

### Preparative Example 15 (Analog 9)

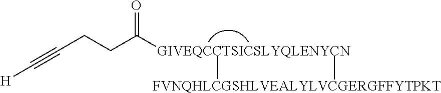

The title compound was synthesized according to General Method C. UPLC-MS Method C: Rt=4.08 min, m/z=1472.46 [(M+4)/4].

### Preparative Example 16 (Analog 10)

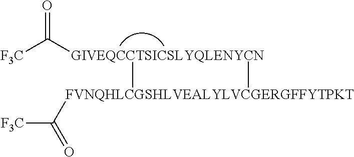

To a suspension of RHI (3.0 g, 0.517 mmol) in DMF (90 mL), was added DIPEA (3.6 mL, 20.61 mmol) and the resulting mixture was stirred at rt for 30 minutes to allow to dissolve as much of the material as possible. Isopropyl 2,2,2-trifluoroacetate (1.4 mL, 9.94 mmol) was added dropwise to the reaction mixture at 0° C. and stirred for 20 h at same temperature. The crude reaction mixture was precipitated out by addition to 500 mL of IPAC/MTBE mixture (4/1), quenched with acetic acid (1.5 mL, 26.2 mmol) and the crude product was isolated by centrifugation. The pellet was re-dissolved in 25% CH3CN in water and purified by reverse-phase chromatography on C8 phase (Column KROMASIL, C8 10 μm 100 Å, 250×50 mm; solvent A=water/0.05% TFA, solvent B=AcCN/0.05% TFA), flow rate=85 mL/min, gradient B in A 23-33% over 30 min). UPLC-MS Method C, Rt=3.92 min, m/z=1500.45 [(M+4)/4].

### Preparative Example 17 (Analog 11)

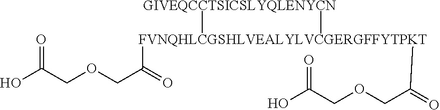

The title compound was synthesized according to General Method B using diglycolic anhydride as the Capping Reagent. UPLC-MS Method C, Rt=3.68 min, m/z=1510.644 [(M+4)/4].

### Preparative Example 18 (Analog 12)

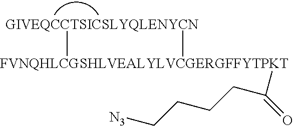

The title compound was synthesized according to General Method A using 2,5-dioxopyrrolidin-1-yl 5-azidopentanoate as the acylating reagent. UPLC-MS Method C, Rt=4.10 min, m/z=1483.886 [(M+4)/4].

### Preparative Example 19 (Analog 13)

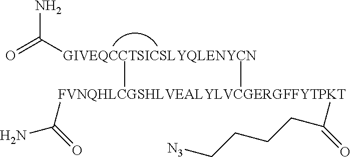

The title compound was prepared by acylation of Analog 1 according to General Method A, using 2,5-dioxopyrrolidin-1-yl 5-azidopentanoate as the acylation reagent. UPLC-MS Method A, Rt=0.88 min, m/z=1505.94 [(M+4)/4].

### Preparative Example 20] (Analog 14)

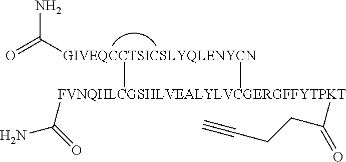

The title compound was prepared by acylation of Analog 1 according to General Method A, using 2,5-dioxopyrrolidin-1-yl pent-4-ynoate as the acylation reagent. UPLC-MS Method A, Rt=0.87 min, m/z=1494.69 [(M+4)/4].

### Preparative Example 21 (Analog 15)

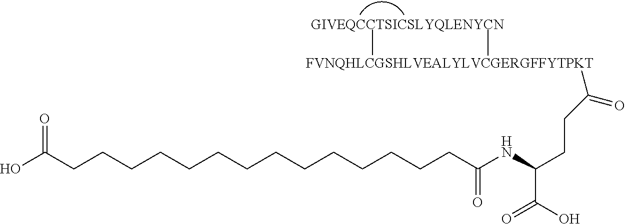

The title compound was prepared according to known procedures for insulin Degludec (WO2010/029159 A1), except that RHI (instead of Des-B30-RHI) was used as the starting material. UPLC-MS Method C, Rt=2.99 min, m/z=1552.367 [(M+4)/4].

### Preparative Example 22 (Analog 16)

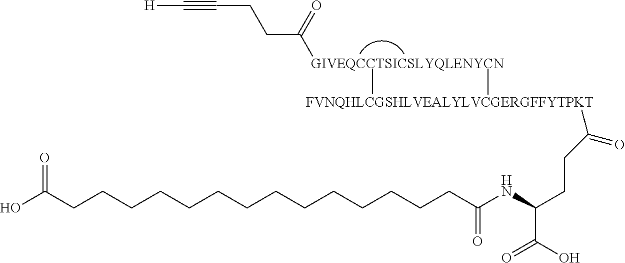

To a 40 mL scintillation vial was added Analog 15 (102.7 mg, 0.017 mmol), 10.0 mL of DMSO and 2,2,6,6-tetramethylpiperidine (0.056 mL, 0.331 mmol). The mixture was allowed to stir at room temperature until the solution became clear (˜30 min). In a separate 20 mL scintillation vial, 2,5-dioxopyrrolidin-1-yl pent-4-ynoate (3.23 mg, 0.017 mmol) was dissolved in 500 μL of DMSO. To the insulin derivative, was added NHS-ester solution in three portions and the progress of the reaction was checked using UPLC-MS. After storing at 4° C. overnight, the reaction mixture was re-dissolved in 30% AcCN in water pH 3 and the resulting solution pH was adjusted to ˜3 and then purified by chromatography on C8 phase (Column KROMASIL, C8 10 μm 100 Å, 250×50 mm; solvent A=water/0.05% TFA, solvent B=AcCN/0.05% TFA), flow rate=85 mL/min, gradient B in A 29-40% over 30 min). The desired fractions were combined and freeze-dried to give the title compound as a white powder. UPLC-MS Method C, Rt=4.27 min, m/z=1571.55 [(M+4)/4].

### General Method D. Synthesis of A1-B29′ Dimer Using bis-NHS Linker

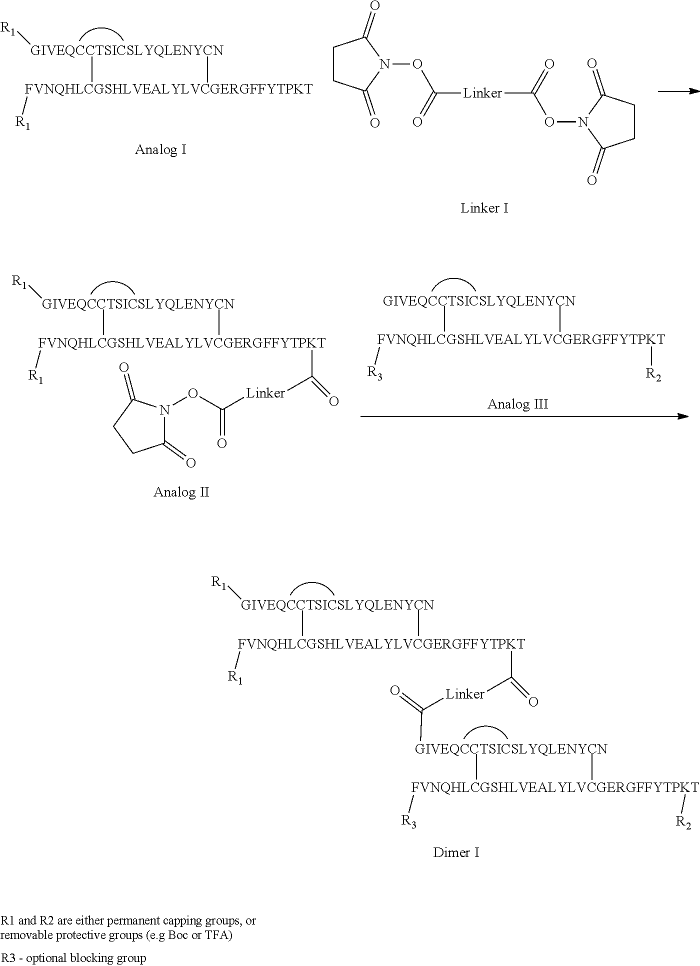

General Method D uses an insulin analog, designated as Analog I in the Scheme, which has A1 and B1 sites blocked by R1 groups, which can be either permanent capping groups or temporary (removable) protective groups. Analog I reacts with excess of bis-NHS linker, which is designated in the Scheme as Linker I, in the presence of an excess of organic base, such as triethylamine, using DMSO or another appropriate organic solvent. The resulting Analog II is a reactive insulin derivative containing an active NHS ester. In order to suppress the side reaction resulting in self-dimerization of Analog I, reverse addition of the reagents can be used (that is, solution of Analog I is added to a premixture of Linker I and organic base), and an excess of Linker I is maintained throughout the reaction. Analog II was found to be sufficiently stable for isolation from the reaction mixture either by precipitation using a “weak” organic solvent such ether, MTBE, IPAC, or a mixture of these solvents, or by reverse-phase chromatography using AcN and water in the presence of 0.05% TFA as a modifier.

In the second step of the synthesis, Analog II reacts with Analog III. Analog III is designed so that it has an R2 group capping B29 position. R2 can be a permanent capping group or temporary (removable) protective group. Optionally, Analog III may have an R3 group capping B1 position. R3 can be a permanent capping group or temporary (removable) protective group. The coupling of Analog II and Analog III is conducted in DMSO as the solvent, in the presence of an organic base, such as triethylamine.

After the coupling of Analog II and Analog III, resulting Dimer I is isolated from the reaction mixture either by precipitation using a “weak” organic solvent such ether, MTBE, IPAC, or a mixture of these solvents, or by reverse-phase chromatography using AcN and water in the presence of 0.05% TFA as a modifier, or by a combination of precipitation and chromatography

The following examples were prepared using General Method D, modifying reagents and reaction conditions as necessary.

### Example 1 (Dimer 1)

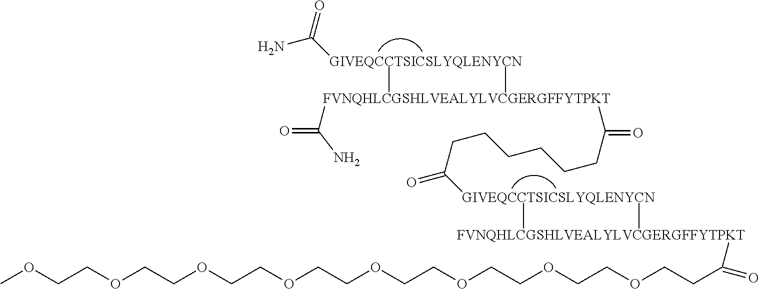

### Step A Synthesis of N^{2,1A},N^{2,1B}-bis(carbamoyl)-N^{6,29B}-(8-((2,5-dioxopyrrolidin-1-yl)oxy)-8-oxooctanoyl) Human Insulin

To a solution of the linker disuccinimidyl suberate (937 mg, 2.55 mmol) in DMSO (10.6 mL) was added triethylamine (236 μl, 1.697 mmol) followed by dropwise addition with stirring of a solution of Analog 1 (1000 mg, 0.170 mmol) in DMSO (10.6 mL). After addition was complete (15 min), the reaction mixture was stirred for another 30 min. The reaction mixture was added into 50 ml of water-20% AcN-0.05% TFA, dropwise with ice cooling (internal temperature not exceeded 20° C.), and pH maintained at 2.5-3 by addition of 1M HCl. The product was purified by chromatography (KROMASIL C8 250×50 mm, 10 μm, 100 Å column; Buffer A: 0.05% TFA in deionized water; Buffer B: 0.05% TFA in AcCN, Flow rate 85 mL/min, gradient B in A 26-40% in 30 min). After lyophilization of fractions, the product was obtained as a white solid, UPLC-MS Method C, Rt=3.90 min, m/z=1537.52 [(M+4)/4].

### Step B. Dimer 1

To a mixture of Analog 3 (80 mg, 0.013 mmol) and the product of Step 1 (81 mg, 0.013 mmol) in 1.0 mL of DMSO was added triethylamine (72.6 μl, 0.521 mmol) and the mixture was stirred for 1 hour. The reaction mixture was added to a mixture of 20% AcN-water-0.05% TFA (10 mL) and removed most of DMSO by 3 cycles of diafiltration in 10 K Amicon tubes with addition of fresh water after each cycle. The product was purified by ion-exchange chromatography (IEC) (PolySULFOETHYL A column, PolyLC Inc., 250×21 mm, 5 μm, 1000 Å; Buffer A: 0.025% (v/v) H3PO4/25% AcCN; Buffer B: 0.025% (v/v) H3PO4/25% AcCN/0.5 M NaCl). The product was re-purified by reverse phase HPLC (Kromasil C8, 250×50 mm, 10 μm, 100 Å; Buffer A: 0.05-0.1% TFA in water; Buffer B: 0.05-0.1% TFA in AcCN, gradient B in A 27-34% in 45 minutes). Obtained as a white solid after lyophilization, UPLC-MS Method C, Rt=3.65 min, m/z=1749.63 [(M+7)/7].

### Example 2 (Dimer 2)

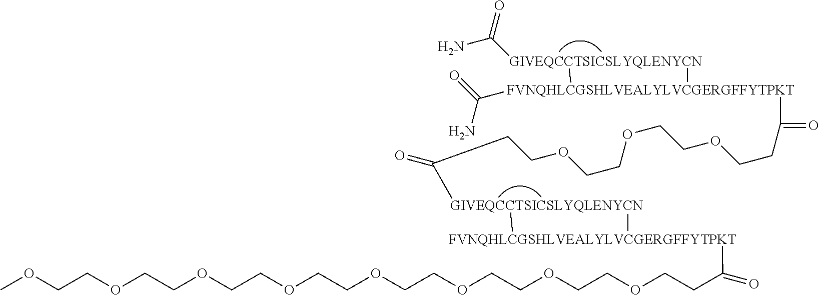

Following steps for the synthesis of Dimer 1 and utilizing bis(2,5-dioxopyrrolidin-1-yl) 3,3′-((oxybis(ethane-2,1-diyl))bis(oxy))dipropionate as the linker, Dimer 2 was obtained. UPLC-MS Method C, Rt=3.76 min, m/z=1759.34 [(M+7)/7].

### Example 3 (Dimer 3)

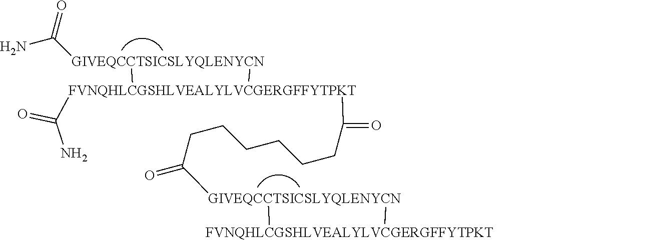

### Step A: Dimerization

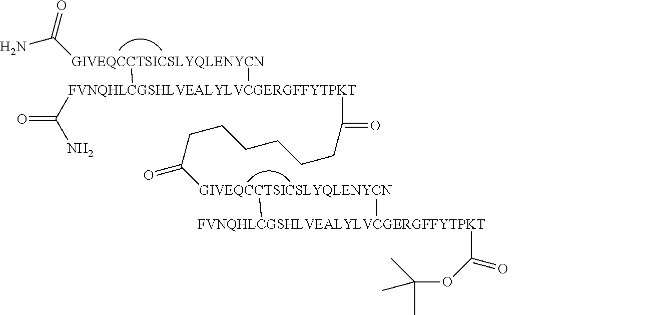

Analog 1 and Analog 4 were dimerized under conditions of General Method D using bis(2,5-dioxopyrrolidin-1-yl) octanedioate as the linker. The product was purified by reverse phase HPLC (Kromasil C8 250×50 mm, 10 μm, 100 Å column; Buffer A: 0.05-0.1% TFA in water; Buffer B: 0.05-0.1% TFA in AcCN, gradient B in A 27-34% in 45 minutes) the resulting product was obtained as a solid after lyophilization, UPLC-MS Method C, Rt=3.90 min, m/z=1706.17 [(M+7)/7].

### Step B: Removal f Boc Protecting Group

The product of Step A was dissolved in 3.0 mL of anhydrous TFA and stirred the solution for 20 min. The reaction mixture was poured in 40 mL of IPAC and the precipitate was collected by centrifugation. The precipitate was washed with 40 mL of diethyl ether (Et2O) and centrifugation was repeated. The pellet was pumped on high vacuum for 30 min. The product was purified by ion-exchange chromatography (IEC) (PolySULFOETHYL A column, PolyLC Inc., 250×21 mm, 5 μm, 1000 Å; Buffer A: 0.025% (v/v) H3PO4/25% AcCN; Buffer B: 0.025% (v/v) H3PO4/25% AcCN/0.5 M NaCl). The product was re-purified by reverse-phase chromatography (Kromasil, C8 10 μM 100 Å, 250×50 mm column; solvent A=water/0.05%TFA, solvent B=AcN/0.05% TFA), Flow=85 mL/min, gradient B in A 27-34% in 45 min. The product was obtained as a solid after lyophilization of fractions. UPLC-MS Method C, Rt=3.68 min, m/z=1974.01 [(M+6)/6].

### Example 4 (Dimer 4)

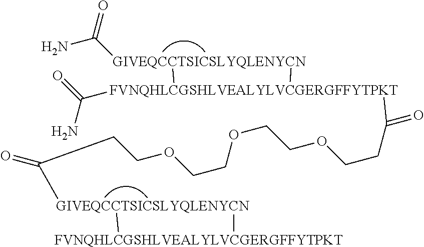

Dimer 4 was synthesized analogously to Dimer 3, except that bis(2,5-dioxopyrrolidin-1-yl) 3,3′-((oxybis(ethane-2,1-diyl))bis(oxy))dipropionate was used as the linker. UPLC-MS Method C, Rt=3.66 min, m/z=1987.06 [(M+6)/6].

### Example 5 (Dimer 5)

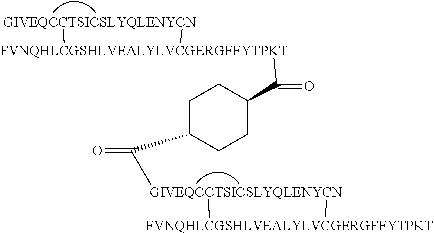

### Step A: TFA/Boc Protected Dimer

Using conditions of General Method D, Analog 10 and Analog 4 were coupled using Linker 1 as the linker. UPLC-MS Method C, Rt=4.04 min, m/z=1721.449 [(M+7)/7].

### Step B TFA Deprotection

The product of Step A (287 mg, 0.024 mmol) was dissolved in 5.0 mL of 10% CH₃CN in H₂O, then 5.0 mL of concentrated NH₄OH (28%) was added dropwise at 0° C. The mixture was stirred at same temperature for 48 hours. The mixture was diluted with 20.0 mL of water pH=3.00) then was diafiltrated using 10K Amicon membrane centrifuge tubes adding fresh water after each cycle to remove the total of 50 mL of permeate. The pH was adjusted to 3.0 by adding 3N HCl and the resultant mixture lyophilized to obtain B29′ Boc-protected dimer insulin intermediate, UPLC-MS Method C, Rt=3.78 min, m/z=1976.285 [(M+6)/6].

### Step C Removal of Boc Protecting Group

The product of Step 2 was dissolved in 2.0 mL of TFA and the resulting mixture was stirred at 0° C. for 10 minutes. The reaction mixture was added dropwise to a centrifuge tube containing 50 mL of MTBE, and the precipitate was isolated by centrifugation. The pellet was rinsed with IPAc (3×20 mL), dried under vacuum for 1 h, and the product was purified by chromatography (KROMASIL C8 250×50 mm, 10 μm, 100 Å column; Buffer A: 0.05% TFA in deionized water; Buffer B: 0.05% TFA in AcCN, Flow rate 85 mL/min, gradient B in A 29-35% in 30 min). UPLC-MS Method C, Rt=3.60 min, m/z=1959.39 [(M+6)/6].

### Example 6 (Dimer 6)

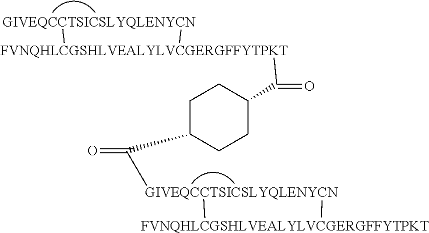

Dimer 6 was synthesized analogously to Dimer 5, except that Linker 2 was used. UPLC-MS Method C, Rt=3.64 min, m/z=1959.04 [(M+6)/6].

### Example 7 (Dimer 7)

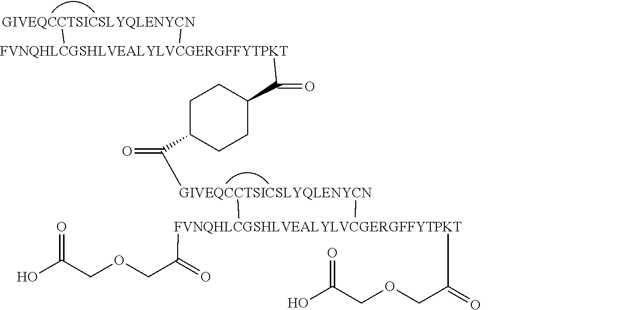

Analog 10 and Analog 11 were coupled using Linker 1 using procedures of General Method D, followed by cleavage of TFA protective group as described for the synthesis of Dimer 5 (step B).

### Example 8 (Dimer 8)

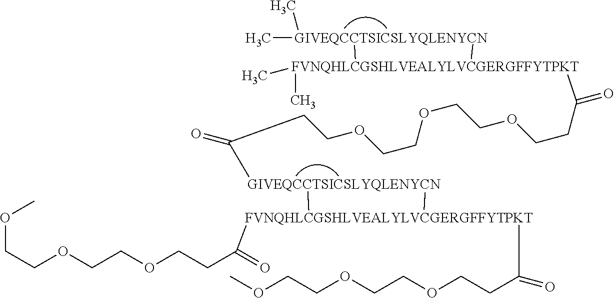

The compound was synthesized from Analog 2 and Analog 7 using bis(2,5-dioxopyrrolidin-1-yl) 3,3′-((oxybis(ethane-2,1-diyl))bis(oxy))dipropionate as the linker and procedures of General Method D. UPLC-MS Method C, Rt=3.80 min, m/z=1748.78 [(M+7)/7].

### Example 9 (Dimer 9)

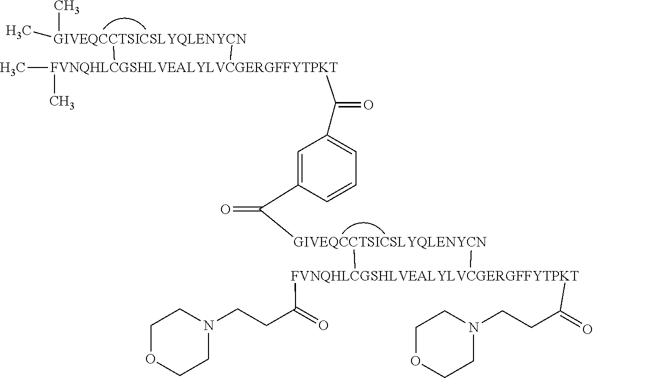

Starting from Analog 2 and Analog 6 and using Linker 3 as the linker, Dimer 9 was synthesized using procedures of General Method D. UPLC-MS Method C, Rt=3.63 min, m/z=1726.95 [(M+7)/7].

### General Method E. Synthesis of Insulin Dimers Using Cu²⁺-Catalyzed Click Chemistry

In an appropriately sized container, appropriate acetylene containing insulin intermediate (Analog) was dissolved, with gentle stirring, at room temperature in a mixed solvent of DMSO and aq. triethylammonium acetate buffer (pH 7.0, concentration 0.2 mM). In another appropriately sized container, appropriate azido containing insulin intermediate (Analog) was dissolved, with gentle stirring, at rt in a mixed solvent of DMSO and water. Both solutions were combined, thoroughly mixed, and degassed by gentle bubbling of nitrogen. To the resulting solution was added freshly prepared sodium ascorbate or ascorbic acid solution (final concentration is 0.5 mM) and, after thoroughly mixing, a solution of 10 mM of CuSO₄ and tris[(1-benzyl-1H-1,2,3-triazol-4-yl)methyl]amine (i.e., TBTA ligand) in 55% DMSO. After degassing by gentle bubbling N₂ and thorough mixing, the mixture was stored at rt, with occasional mixing, overnight. The reaction mixture was carefully diluted with a mixed solvent (v/v 7:3 AcCN/water with 0.05% TFA) at 0° C. and pH was adjusted to 2.5 using 0.1, 1.0 N HCl (and 0.1 N NaOH if needed). The solution was first concentrated by ultrafiltration, either through a tangential flow filtration (TFF) system or using Amicon Ultra-15 Centrifugal Units, with 1K, 3K, or 10K MWCO membrane. The concentrated solution was usually first subjected to ion exchange chromatography (PolySULFOETHYL A column, PolyLC Inc., 250×21 mm, 5 μm, 1000 Å; Buffer A: 0.1% (v/v) H₃PO₄/25% AcCN; Buffer B: 0.1% (v/v) H₃PO₄/25% AcCN/0.5 M NaCl). Fractions containing desired product with desired purity were combined and concentrated using TFF system or Amicon Ultra-15. The resulting solution was then further purified by reverse phase HPLC (Waters C4 250×50 mm column, 10 μm, 1000 Å column or KROMASIL C8 250×50 mm, 10 μm, 100 Å column; Buffer A: 0.05-0.1% TFA in water; Buffer B: 0.05-0.1% TFA in AcCN). Fractions containing the desired product with desired purity were combined and freeze-dried or buffer exchanged using TFF system and/or Amicon Ultra-15 to give the insulin dimers.

The following examples were prepared using General Method E, modifying reagents and reaction conditions as necessary

### Example 10 (Dimer 10)

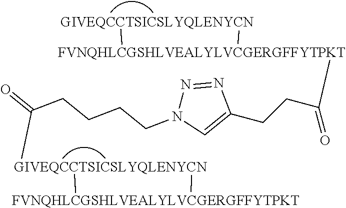

Analog 5 (60 mg, 10.19 μmol) was dissolved in a mixture of 5.6 mL DMSO and 5.6 mL of water and added 3.0 mL of 0.2 mM triethylammonium acetate buffer (pH=7.0). In a separate vial, Analog 8 (60.5 mg, 10.19 μmol) dissolved in a mixture of 5.6 mL DMSO and 5.6 mL water. These two solutions were mixed and treated with 3.0 mL of freshly prepared 5 mM ascorbic acid solution (0.5 mL). The reaction mixture was degassed by nitrogen bubbling for 1 minute, and treated with 1.5 ml of 10 mM Cu(II)-TBTA solution in 55% DMSO-Water. The mixture was shaken for 3 hours and left standing over night. The resultant precipitate was dissolved in 10 volumes of 30% AcN-Water-0.1% TFA, pH adjusted to 2.5 with 1N HCl, and the solution was concentrated in Amicon tubes. The product was isolated by reverse-phase chromatorgraphy (Kromasil C8 250×50 mm, 10 μm, 100 Å column; Buffer A: 0.05-0.1% TFA in water; Buffer B: 0.05-0.1% TFA in AcCN). Fractions containing the title conjugate were combined and freeze-dried to give the title product as white powder. UPLC-MS Method C: Rt=3.16 min, m/z=1970.94[(M+6)/6].

### Example 11 (Dimer 11)

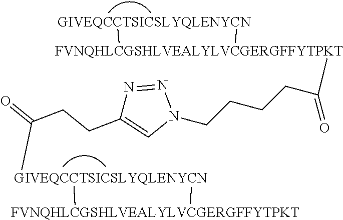

Dimer 11 was obtained from Analog 9 and Analog 12 using procedures of General Method E. UPLC-MS Method C: Rt=3.70 min, m/z=1971.13 [(M+6)/6].

### Example 12 (Dimer 12)

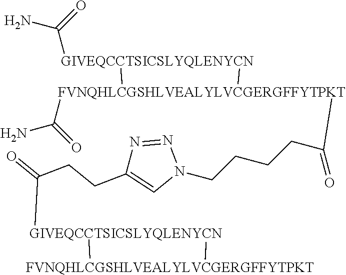

Dimer 12 was obtained from Analog 9 and Analog 13 using procedures of General Method E. UPLC-MS Method C: Rt=3.72 min, m/z=1985.20 [(M+6)/6].

### Example 13 (Dimer 13)

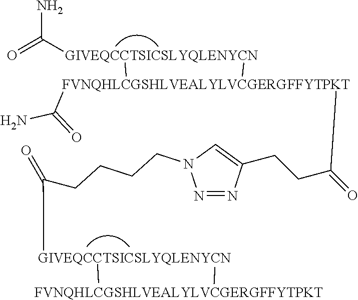

Dimer 13 was obtained from Analog 8 and Analog 14 using procedures of General Method E. UPLC-MS Method C: Rt=3.72 min, m/z=1985.24 [(M+6)/6].

### Example 14 (Dimer 14)

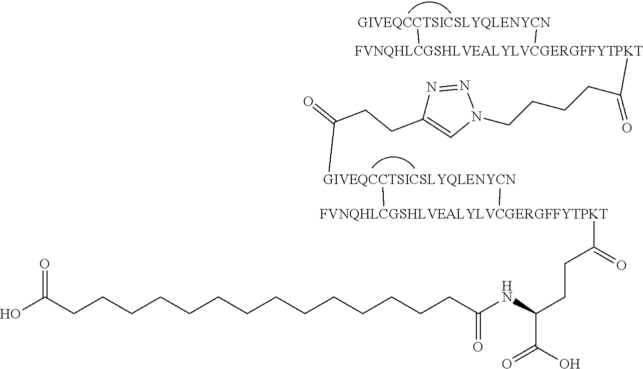

Dimer 14 was obtained from Analog 12 and Analog 16 using procedures of General Method E. UPLC-MS Method C: Rt=4.03 min, m/z=1746.02 [(M+7)/7].

### Example 15 (Dimer 15

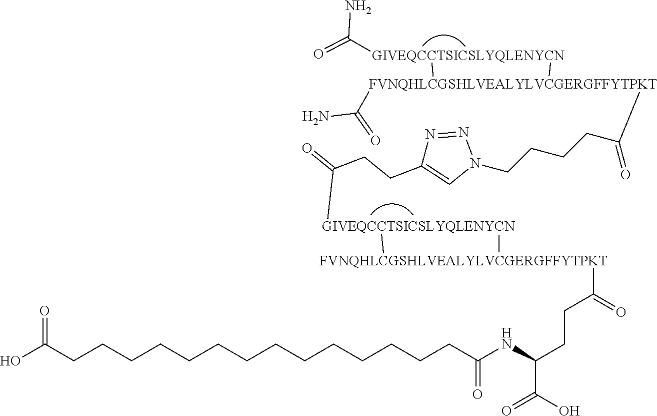

Dimer 15 was obtained from Analog 13 and Analog 16 using procedures of General Method E. UPLC-MS Method C: Rt=4.14 min, m/z=1758.91 [(M+7)/7].

The following compounds in were obtained using the procedure analogous to General Method E but substituting appropriate starting materials that are either commercially available or prepared using procedure analogous to those described in Examples 10 through 15

| | | | | UPLC- |
|---|---|---|---|---|
| | | Rt | (M + 6)/6 or | MS |
| Dimer | Description | (min) | ((M + 7)/7) | Method |
| 16 | 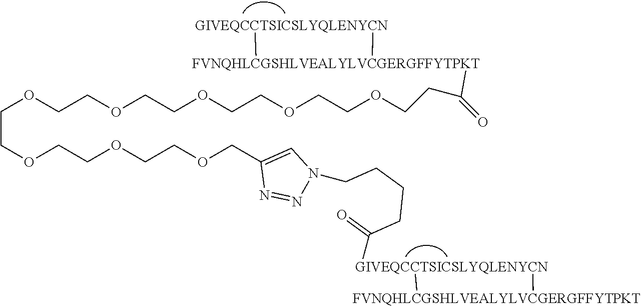 | 3.41 | 1737.71 | C |
| 17 | 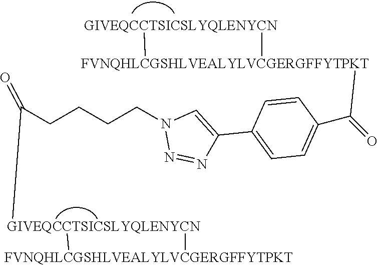 | 3.38 | 1696.77 | C |
| 18 | 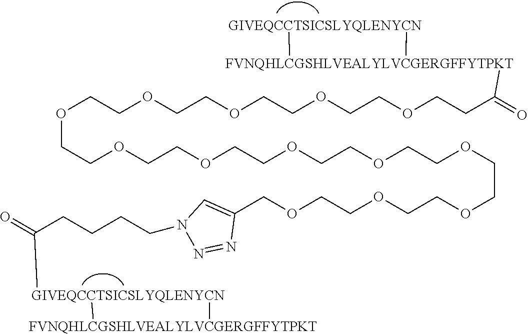 | 3.41 | 1769.61 | C |
| 19 | 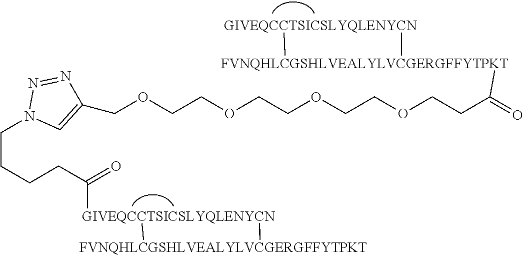 | 3.38 | 1712.82 | C |
| 20 | 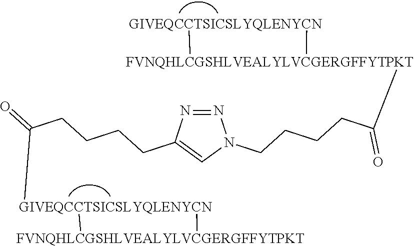 | 3.56 | 1975.49 | C |
| 21 | 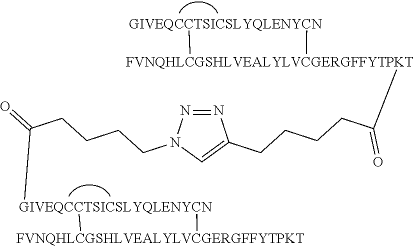 | 3.36 | 1975.63 | C |
| 22 | 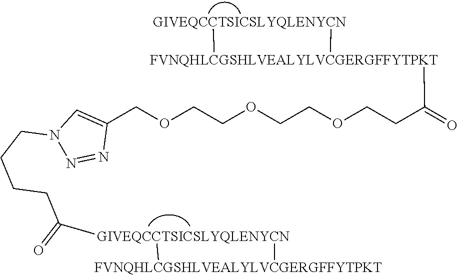 | 3.34 | 1990.42 | C |
| 23 | 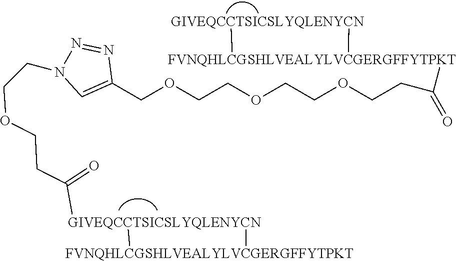 | 3.12 | 1993.488 | C |
| 24 | 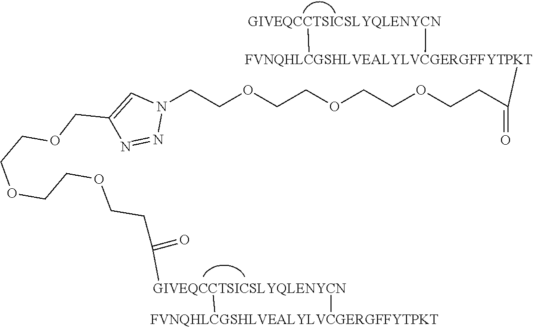 | 3.48 | 1721.37 | C |
| 25 | 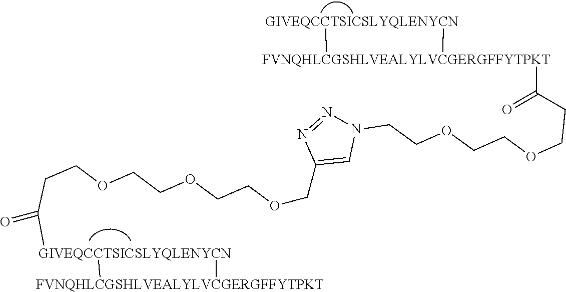 | 3.61 | 1715.02 | C |
| 26 | 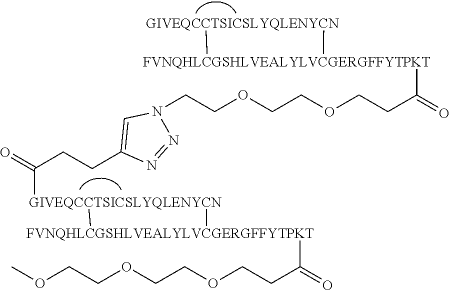 | 3.78 | 1723.16 | C |
| 27 | 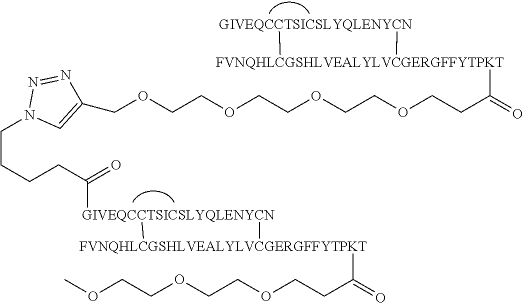 | 2.67 | 1735.70 | D |
| 28 | 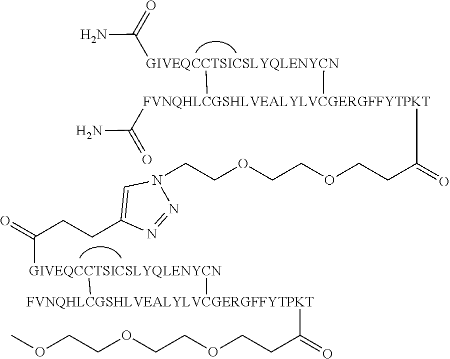 | 3.79 | 1734.90 | |
| 29 | 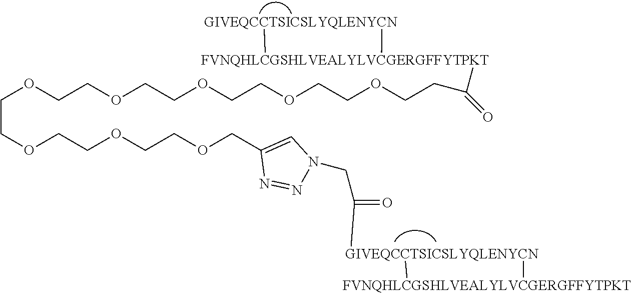 | 0.83 | 1724.58 | A |
| 30 | 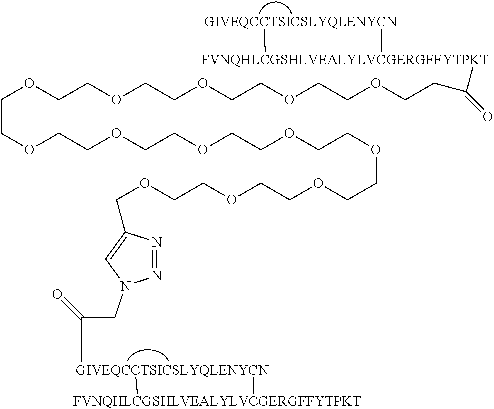 | 0.83 | 1756.52 | A |
| 31 | 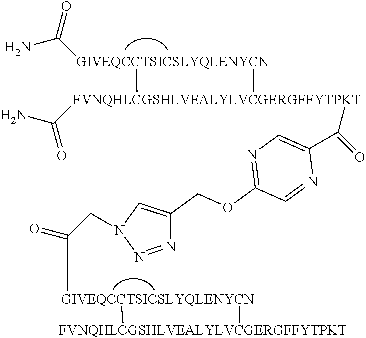 | 0.87 | 1982.93 | A |
| 32 | 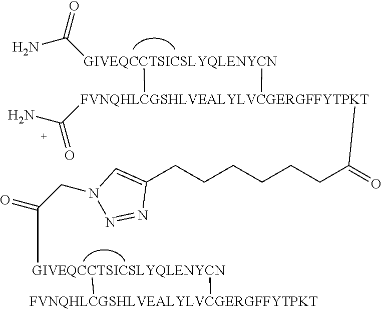 | 0.88 | 1979.13 | A |
| 33 | 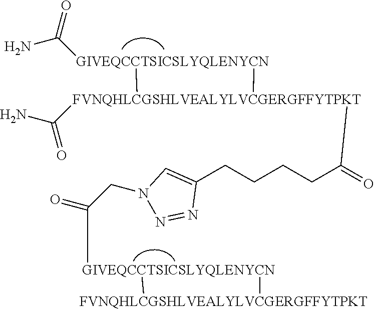 | 1.49 | 1974.01 | D |

### Example 16

### A. Insulin Receptor Binding Assays were Performed as Follows

IR binding assay was run in a scintillation proximity assay (SPA) in 384-well format using cell membranes prepared from CHO cells overexpressing human IR(B) grown in F12 media containing 10% FBS and antibiotics (G418, Penicillin/Strepavidin). Cell membranes were prepared in 50 mM Tris buffer, pH 7.8 containing 5 mM MgCl₂. The assay buffer contained 50 mM Tris buffer, pH 7.5, 150 mM NaCl, 1 mM CaCl₂, 5 mM MgCl₂, 0.1% BSA and protease inhibitors (Complete-Mini-Roche). Cell membranes were added to WGA PVT PEI SPA beads (5 mg/mL final concentration) followed by addition of insulin dimer molecules at appropriate concentrations. After 5-15 min incubation at room temperature, ¹²⁵[I]-insulin was added at 0.015 nM final concentration for a final total volume of 50 μL. The mixture was incubated with shaking at room temperature for 1 to 12 hours followed by scintillation counting to determine ¹²⁵[I]-insulin binding to IR and the titration effects of insulin dimer molecules on this interaction.

### B. Insulin Receptor (IR) AKT-Phosphorylation Assays were Performed as Follows

Insulin receptor activation can be assessed by measuring phosphorylation of the Akt protein, a key step in the insulin receptor signaling cascade. CHO cell lines overexpressing human IR were utilized in a Homogeneous Time Resolved Fluorescence (HTRF) sandwich ELISA assay kit (Cisbio “Phospho-AKT(Ser473) and Phospho-AKT(Thr308) Cellular Assay Kits”). Cells were grown in F12 media supplemented with 10% fetal bovine serum (FBS), 400 mg/mL Geneticin (G418) and 10 mM HEPES (4-(2-hydroxyethyl)-1-piperazineethanesulfonic acid). Prior to assay, the cells were incubated in serum free media for 2 to 4 hr. Alternatively, the cells could be frozen and aliquoted ahead of time in media containing 20% DMSO and used in the assay upon thawing, spin down and re-suspension. Cells were plated at 10,000 cells per well in 20 mL of the serum free F12 media in 384-well plates. Humulin and insulin glargine controls were run on each plate of test compounds. The titrated compounds were added to the cells (2 mL per well, final concentrations=1000 nM titrated down to 0.512 pM in 1:5 fold dilutions) and incubated at 37° C. for 30 min. The cells were lysed with 8 mL of the prepared lysis buffer provided in the CisBio kit and incubated at 25° C. for 1 hr. The diluted antibody reagents (anti-AKT-d2 and anti-pAKT-Eu3/cryptate) were prepared according to the kit instructions and then 10 mL was added to each well of cell lysate followed by incubation at 25° C. for 3.5 to 5 hr. The plate was read by in an Envision plate reader (Excitation=320 nm; Emission=665 nm) to determine the IR pAkt agonist activity with regard to both potency and maximum response for each compound.

Table V shows the in vitro biological activity of the insulin dimers towards the insulin receptor (IR). The activities were measured by either ligand competition assays as described in Example 16A or functional AKT-phosphorylation assay as described in Example 16B.

TABLE V

| Dimer | IR Binding | IRpAkt | IRpAkt |
|---|---|---|---|
| No. | IC50 (nM) | EC50 (nM) | % Act |
| 1 | 6.5 | 0.183 | 33 |
| 2 | 2.0 | 0.213 | 30 |
| 3 | 1.7 | 0.220 | 38 |
| 4 | 2.6 | 0.010 | 29 |
| 5 | 0.7 | 0.077 | 27 |
| 6 | 0.7 | 0.094 | 27 |
| 7 | 1.8 | 0.068 | 16 |
| 8 | 2.1 | 0.146 | 19 |
| 9 | 1.1 | 0.134 | 31 |
| 10 | 0.6 | 0.046 | 30 |
| 11 | 1.6 | 0.073 | 21 |
| 12 | 5.9 | 0.001 | 49 |
| 13 | 10.9 | 0.302 | 30 |
| 14 | 42.2 | 3.994 | 51 |
| 16 | 6.5 | 0.331 | 61 |
| 17 | 0.8 | 0.021 | 30 |
| 18 | 7.9 | 0.150 | 84 |
| 19 | 3.1 | 0.074 | 40 |
| 20 | 1.1 | 0.095 | 28 |
| 21 | 0.9 | 0.361 | 85 |
| 22 | 2.4 | 0.073 | 30 |
| 23 | 0.7 | 0.125 | 38 |
| 24 | 2.5 | 0.428 | 35 |
| 25 | 3.8 | 0.464 | 35 |
| 26 | 2.7 | 0.101 | 31 |
| 27 | 2.1 | 0.147 | 38 |
| 28 | 9.7 | 0.107 | 27 |
| 29 | 1.2 | 0.149 | 35 |
| 30 | 4.0 | 0.359 | 61 |
| 31 | 2.2 | 0.105 | 19 |
| 32 | 49.6 | 0.384 | 38 |
| 33 | 8.5 | 0.154 | 26 |

### Example 17

The glucose lowering effect of Dimers 2, 5, 7, 11, and 20 were compared to RHI in Diabetic Yucatan miniature pigs (D minipigs) as follows.

Yucatan minipigs were rendered Type 1 diabetic by Alloxan injections following a proprietary protocol developed by Sinclair Research Center (Auxvasse, MO). Induction is considered successful if basal glucose levels exceed 150 mg/dL. Diabetic(D) minipigs with plasma glucose levels of approximately 300 mg/dl were utilized in these experiments.

Male Yucatan minipigs, instrumented with two Jugular vein vascular access ports (VAP), were used in these studies. On the day of the study after an overnight fast, minipigs were placed in slings, and VAPs were accessed for infusion and sampling. At t=0 min, and after collecting two baseline blood samples for plasma glucose measurement (t=−30 minutes and t=0 minutes), minipigs were administered Humulin (recombinant human insulin, RHI) or insulin dimer (i.e., 2, 5, 7, 11, or 20) as a single bolus IV, at 0.69 nmol/kg. Humulin and the immediately preceding aforementioned insulin dimers were formulated at 69 nmol/ml in a buffer containing Glycerin, 16 mg/mL; Metacresol, 1.6 mg/mL; Phenol, 0.65 mg/mL; Anhydrous Sodium Phosphate, Dibasic, 3.8 mg/mL; pH adjusted to 7.4 with HCl. After dosing, sampling continued for 480 minutes; time points for sample collection were −30 min, 0 min , 8 min, 15 min, 30 min, 45 min, 60 min, 90 min, 120 min, 150 min, 180 min, 210 min, 240 min, 270 min, 300 min, 330 min, 360 min, 420 min, 480 min. Blood was collected in K3-EDTA tubes, supplemented with 10 μg/mL aprotinin, and kept on ice until processing, which occurred within 30 minutes of collection. After centrifugation at 3000 rpm, 4° C., for 8 min, plasma was collected and aliquoted for glucose measurement using a Beckman Coulter AU480 Chemistry analyzer and for compound levels measurement.

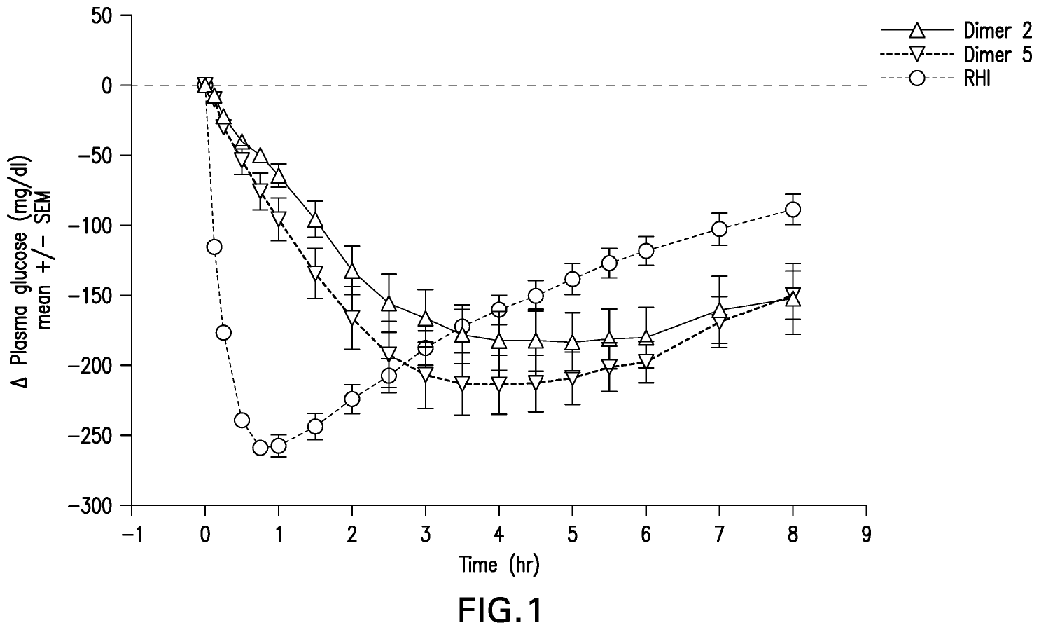

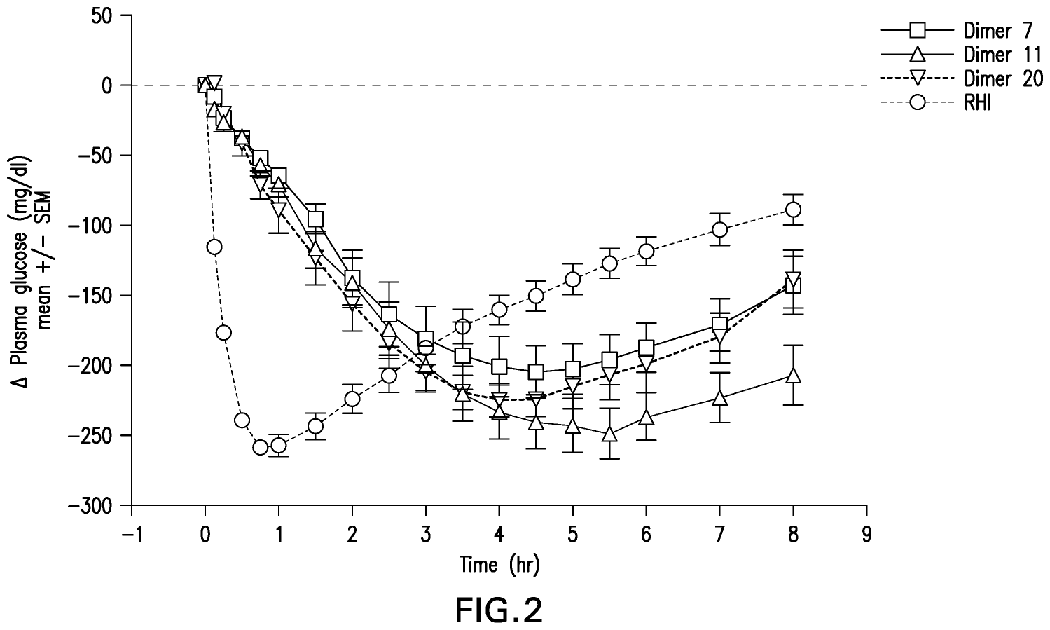

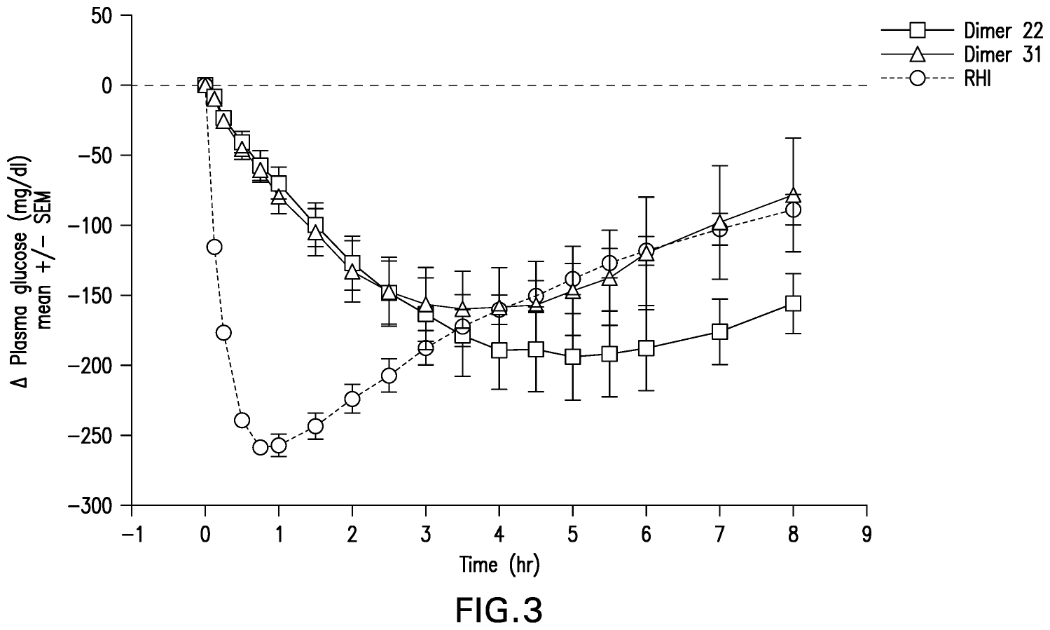

The results are shown in FIGS. 1-3. The results are presented as the change of glucose at any given time point to time 0 and show that the insulin dimers present less risk of promoting hypoglycemia than RHI.

Below are representable sequences useful in the claimed invention.

Table of Sequences

| SEQ ID | | |
|---|---|---|
| NO: | Description | Sequence |
| 1 | Homo sapiens insulin A chain | GIVEQCCTSICSLYQLENYCN |
| 2 | Homo sapiens insulin B chain | FVNQHLCGSHLVEALYLVCG |
| | | ERGFFYTPKT |
| 3 | Artificial sequence insulin A | GX₂X₃EQCCX&SICSLYQLX₁₇NX₁₉ |
| | chain X₂ is isoleucine or | CX₂₃ |
| | threonine; | |
| | X₃ is valine, glycine, or | |
| | leucine; | |
| | X₈ is threonine or histidine; | |
| | X₁₇ is glutamic acid or | |
| | glutamine; | |
| | X₁₉ is tyrosine, 4-methoxy- | |
| | phenylalanine, alanine, or 4- | |
| | amino phenylalanine; | |
| | X₂₃ is asparagine or glycine; | |
| 4 | Artificial sequence insulin B chain | X₂₅LCGX₂₉X₃₀LVEALYLVCGERG |
| | X₂₅ is histidine or threonine; | FX₂₇YTX₃₁X₃₂ |
| | X₂₇ is phenylalanine or aspartic acid; | |
| | X₂₉ is alanine, glycine or serine; | |
| | X₃₀ is histidine, aspartic acid, | |
| | glutamic acid, homocysteic acid, or | |
| | cysteic acid; | |
| | X₃₁ is aspartic acid, proline or lysine; | |
| | and | |
| | X₃₂ is proline or lysine, with the | |
| | proviso that at least one of X₃₁ or | |
| | X₃₂ is lysine | |
| 5 | Artificial sequence insulin B chain | X₂₂VNQX₂₅X₂₆CGX₂₉X₃₀LVEALY |
| | X₂₂ is phenylalanine or desamino- | LVCGERGFX₂₇YTX₃₁X₃₂X₃₃X₃₄ |
| | phenylalanine; | X₃₅ |
| | X₂₅ is histidine or threonine; | |
| | X₂₆ is glycine or leucine; | |
| | X₂₇ is phenylalanine or aspartic acid; | |
| | X₂₉ is alanine, glycine, or serine; | |
| | X₃₀ is histidine, aspartic acid, | |
| | glutamic acid, homocysteic acid, or | |
| | cysteic acid; | |
| | X₃₁ is aspartic acid, proline, or | |
| | lysine; | |
| | X₃₂ is lysine or proline; | |
| | X₃₃ is threonine, alanine, or absent; | |
| | X₃₄ is arginine or absent; and | |
| | X₃₅ is arginine or absent; | |
| | With the proviso at least one of | |
| | X₃₁ or X₃₂ is lysine | |
| 6 | Artificial sequence | FVNQHLCGSHLVEALYLVCGERGF |
| | insulin lispro B chain | FYTKPT |
| 7 | Artificial sequence | GIVEQCCTSICSLYQLENYCG |
| | insulin glargine A chain | |
| 8 | Artificial sequence | FVNQHLCGSHLVEALYLVCGERGF |
| | Insulin glargine B chain | FYTPKTRR |
| 9 | Artificial sequence | FVNQHLCGSHLVEALYLVCGERGF |
| | Insulin aspart B chain | FYTDKT |
| 10 | Artificial sequence | FVNQHLCGSHLVEALYLVCGERGF |
| | B: des30 | FYTPK |

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the claims attached herein.

1. An insulin dimer comprising: an epsilon (ε)-amine of a lysine group on the B chain of a first insulin having a first A-chain polypeptide and first B-chain polypeptide and an α-amino group of A1′ residue of a second insulin molecule having a second A′-chain polypeptide and second B′-chain polypeptide conjugated together by a bifunctional linker moiety selected from Linker 1, Linker 2, Linker 3, Linker 4, Linker 5, Linker 6, Linker 7, Linker 8, Linker 9, Linker 10, Linker 11, Liner 12, Linker 13, Linker 14, Linker 15, Linker 16, Linker 17, Linker 18, Linker 19, Linker 20, Linker 21, Linker 22, and Linker 23.

2. The insulin dimer of claim 1 wherein the linker moiety covalently links the first insulin or insulin analog heterodimer and the second insulin or insulin analog heterodimer via the epsilon amino group of a lysine residue at or near the carboxy terminus of the first insulin or insulin analog heterodimer's B chain polypeptides and the alpha amino group of a glycine at the amino terminus of the second insulin or insulin analog heterodimer's A-chain.

3. The insulin dimer of claim 1 wherein at least one of the first or second A-chain or B-chain polypeptides is conjugated at its N-terminal amino acid to a capping group or at least the N-terminal amino acids of the first insulin heterodimer molecule are conjugated to a capping group or the N-terminal amino acids of both the first insulin heterodimer and second insulin heterodimer are conjugated to a capping group.

4. The insulin dimer of claim 3 wherein the capping group is a linear or branch C₁₋₆ alkyl, or has the general formula RC(O)—, where R is:
a) a peptide,
b) PEG,
c) linear or branched C₁₋₆ alkyl chain,
d) R′NH—, or
e) R′O—,
wherein R′ is H (when R is R′NH—), peptide, PEG, or linear or branched alkyl chain, and wherein each said peptide, PEG and linear or branched alkyl may be unsubstituted or substituted with 1 to 3 groups selected from amino-, phosphono-, hydroxy-, carboxylic acid, amino acid, PEG, and saccharides.

5. The insulin dimer of claim 3, wherein the capping group is
a) dimethyl,
b) isobutyl, or
c) RC(O)— which is selected from acetyl, phenylacetyl, methoxy acetyl, 2-(carboxymethoxy)acetyl, 2-[bis(carboxymethylamino)]acetyl, carbamoyl, N-alkyl carbamoyl, glutaryl, trifluoroacetyl, glycyl, AEG-C6, PEG1, PEG2, PEG3, PEG4, PEG5, PEG8, PEG24, and alkoxycarbonyl.

6. The insulin dimer of claim 5, wherein the capping group is an acetyl, phenylacetyl, carbamoyl, N-alkyl carbamoyl, isobutyl, methoxy acetyl, 2-(carboxymethoxy)acetyl, glutaryl, Me2, carbamoyl, glycyl, AEG-C6, PEG1, PEG2, PEG8, N-dimethyl, and alkoxycarbonyl.

7. The insulin dimer of claim 1, wherein the first insulin and the second insulin heterodimers are independently native human insulin, insulin lispro, insulin aspart, desB30 insulin, or insulin glargine.

8. The insulin dimer of claim 1, wherein each A-chain polypeptide independently comprises the amino acid sequence GX₂X₃EQCCX₃SICSLYQLX₁₇NX₁₉CX₂₃ (SEQ ID NO:3) and each B-chain polypeptide independently comprises the amino acid sequence X₂₅LCGX₂₉X₃₀L VEALYLVCGERGFX₂₇YTX₃₁X₃₂ (SEQ ID NO:4) or X₂₂VNQX₂₅X₂₆CGX₂₉X₃₀LVEALYLVCGERGFX₂₇YTX₃₁X₃₂X₃₃X₃₄X₃₅ (SEQ ID NO:5) wherein X₂ is isoleucine or threonine; X₃ is valine, glycine, or leucine; X₈ is threonine or histidine; X₁₇ is glutamic acid or glutamine; X₁₉ is tyrosine, 4-methoxy-phenylalanine, alanine, or 4-amino phenylalanine; X₂₃ is asparagine or glycine; X₂₂ is or phenylalanine and desamino-phenylalanine; X₂₅ is histidine or threonine; X₂₆ is leucine or glycine; X₂₇ is phenylalanine or aspartic acid; X₂₉ is alanine, glycine, or serine; X₃₀ is histidine, aspartic acid, glutamic acid, homocysteic acid, or cysteic acid; X₃₁ is aspartic acid, proline, or lysine; X₃₂ is lysine or proline; X₃₃ is threonine, alanine, or absent; X₃₄ is arginine or absent; and X₃₅ is arginine or absent; with the proviso at least one of X₃₁ or X₃₂ is lysine.

9. A composition comprising: an α-amino group of A1 residue of a second insulin or insulin analog heterodimer having a first A-chain polypeptide and first B-chain polypeptide and an epsilon (ε)-amine of a lysine group on the B chain of a first insulin or insulin analog heterodimer having a second A-chain polypeptide and second B-chain polypeptide conjugated together by a bifunctional linker moiety selected from Linker 1, Linker 2, Linker 3, Linker 4, Linker 5, Linker 6, Linker 7, Linker 8, Linker 9, Linker 10, Linker 11, Liner 12, Linker 13, Linker 14, Linker 15, Linker 16, Linker 17, Linker 18, Linker 19, Linker 20, Linker 21, Linker 22, and Linker 23;
wherein the insulin analog is selected from insulin lispro, insulin aspart, and insulin glargine; and
wherein the amino terminus of at least one of the A-chain polypeptides and the B-chain polypeptides of the first insulin polypeptide or second insulin polypeptide is covalently linked to a capping group.

10. The composition of claim 9, wherein the first and second insulin or insulin analog heterodimers are the same or wherein the first and second insulin or insulin analog heterodimers are different.

11. The composition of claim 9, wherein the linker moiety covalently links the first insulin or insulin analog heterodimer and the second insulin or insulin analog heterodimer via the epsilon amino group of a lysine residue at or near the carboxy terminus of the first insulin or insulin analog heterodimer's B chain polypeptides and the alpha amino group of a glycine at the amino terminus of the second insulin or insulin analog heterodimer's A-chain.

12. The composition of claim 9, wherein at least one of the first or second A-chain or B-chain polypeptides is conjugated at its N-terminal amino acid to a capping group or at least the N-terminal amino acids of the first insulin heterodimer molecule are conjugated to a capping group or the N-terminal amino acids of both the first insulin heterodimer and second insulin heterodimer are conjugated to a capping group.

13. The composition according to claim 12 wherein the capping group is a linear or branch C₁₋₆ alkyl, or has the general formula RC(O)—, where R is:
a) a peptide,
b) PEG,
c) linear or branched C₁₋₆ alkyl chain,
d) R′NH—, or
e) R′O—,
wherein R′ is H (when R is R′NH—), peptide, PEG, or linear or branched alkyl chain, and wherein each said peptide, PEG and linear or branched alkyl may be unsubstituted or substituted with 1 to 3 groups selected from amino-, phosphono-, hydroxy-, carboxylic acid, amino acid, PEG, and saccharides.

14. The composition of claim 13, wherein the capping group is
a) dimethyl,
b) isobutyl, or
c) RC(O)— which is selected from acetyl, phenylacetyl, methoxy acetyl, 2-(carboxymethoxy)acetyl, 2-[bis(carboxymethylamino)]acetyl, carbamoyl, N-alkyl carbamoyl, glutaryl, trifluoroacetyl, glycyl, AEG-C6, PEG1, PEG2, PEG3, PEG4, PEG5, PEG8, PEG24, and alkoxycarbonyl.

15. A composition comprising an insulin dimer selected from the group consisting of Dimers 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, and 33.

16. The composition of claim 15, wherein the composition further comprises a pharmaceutically acceptable carrier.

17. The composition of claim 15, wherein the composition further comprises a GLP-1 receptor agonist.

18. A method for treating diabetes comprising administering to an individual with diabetes a therapeutically effective amount of a composition comprising the insulin receptor partial agonist of claim 1.

19. The method of claim 18, wherein the diabetes is Type 1 diabetes, Type 2 diabetes, or gestational diabetes.

20. A composition for the treatment of diabetes comprising the insulin receptor partial agonist of claim 9.

21. The composition of claim 20, wherein the diabetes is Type 1 diabetes, Type 2 diabetes, or gestational diabetes.

22. (canceled)

23. (canceled)
